(19) United States
(12) Patent Application Publication
(10) Pub. No.: US 2013/0178463 A1
(43) Pub. Date: Jul. 11, 2013

(54) ENHANCED TRANSBUCCAL DRUG DELIVERY SYSTEM AND COMPOSITIONS

(71) Applicants: Bassam B. Damaj, San Diego, CA (US); Richard Martin, San Diego, CA (US); Bozena Michniak-Kohn, Piscataway, NJ (US); Long-Sheng Hu, Piscataway, NJ (US)

(72) Inventors: Bassam B. Damaj, San Diego, CA (US); Richard Martin, San Diego, CA (US); Bozena Michniak-Kohn, Piscataway, NJ (US); Long-Sheng Hu, Piscataway, NJ (US)

(73) Assignees: Rutgers, The State University of New Jersey, New Brunswick, NJ (US); Nexmed Holdings, Inc., San Diego, CA (US)

(21) Appl. No.: 13/825,823

(22) PCT Filed: Sep. 23, 2011
(86) PCT No.: PCT/US11/01645

§ 371 Date: Mar. 24, 2013

Related U.S. Application Data
(60) Provisional application No. 61/386,001, filed on Sep. 24, 2010.

(51) Int. Cl. A61K 47/18

(52) U.S. Cl. CPC A61K 47/18
USPC 514/211.07; 514/343; 514/397; 514/626

(57) ABSTRACT

A buccal delivery system is disclosed suitable for delivery of a therapeutic agent to the oral cavity of a patient. The delivery system comprises a matrix for containing and releasing the therapeutic agent into the oral cavity and an alkyl N,N-disubstituted amino acetate in said matrix. A particularly preferred delivery system comprises a matrix containing an effective amount of therapeutic agent together with an alkyl N,N-disubstituted amino acetate, such as dodecyl 2-(N,N-dimethylamino) propionate salt.

### CROSS-REFERENCE TO RELATED APPLICATION

This application claims the priority of U.S. Provisional application for patent Ser. No. 61/386,001, filed Sep. 24, 2010, the entire disclosures of which are incorporated herein by reference in its entirety.

### FIELD OF INVENTION

This invention relates to the oral delivery of therapeutic compositions, and more particularly to a buccal delivery system suitable for enhancing transbuccal delivery of a therapeutic agent to the oral cavity of a patient and to oral therapeutic compositions.

### BACKGROUND OF INVENTION

The delivery of a therapeutic agent to the oral cavity of a patient is a desired form of administration. The present invention provides a buccal delivery system comprising a matrix for containing and releasing into the oral cavity a therapeutic agent and a drug-releasing enhancing agent. Also provided are oral therapeutic compositions for transbuccal delivery of a therapeutic agent to the oral cavity of a patient.

### SUMMARY OF INVENTION

Disclosed is an oral composition and buccal delivery system suitable for enhancing delivery of a therapeutic agent to the oral cavity of a patient. The oral composition and oral delivery system comprises a matrix for containing an effective therapeutic amount of therapeutic agent and an alkyl N,N-disubstituted amino acetate as a drug-releasing enhancing agent for enhancing and releasing the therapeutic agent into the oral cavity. Particularly preferred as a penetration enhancing and drug releasing agent is dodecyl 2-(N,N-dimethylamino) propionate salt.

In one preferred embodiment, the matrix comprises a gel composition or a paste composition, preferably included in a transbuccal patch. Another preferred embodiment comprises an orally disintegrating tablet which comprises a matrix containing an effective amount of a therapeutic agent together with an alkyl N,N-disubstituted amino acetate. The tablet is suitable for buccal or sublingual administration of the therapeutic agent.

The therapeutic compositions can comprise a physiologically acceptable carrier for the therapeutic agent, if desired. The dosage and dosage form of the therapeutic agent in any given case depends on the condition being treated, the particular therapeutic agent that is used to treat the condition, as well as the form of buccal administration.

### BRIEF DESCRIPTION OF THE DRAWINGS

FIG. 1 is a graphic representation of the effect of current on flux of transbuccal delivery of ODAN.HCl with data presented as means±S.D. (4≦N≦5).

FIG. 2 is a graphic representation of the effect of iontophoretic current on the cumulative amount of ODAN.HCl permeated through procine buccal tissue at 24 hours with data presented as means±S.D. (4≦N≦5).

FIG. 3 is a graphic representation of the effect of chemical enhancers on the cumulative amount of ODAN.HCl permeated through procine buccal tissue at 24 hours with data presented as means±S.D. (N=4).

FIG. 4 is a graphic representation of the combined treatment of iontophoresis with chemical enhancers on ODAN.HCl permeation through procine buccal tissue at 24 hours with data presented as means±S.D. (3≦N≦5).

FIG. 5 shows the morphology of untreated porcine buccal tissue (EP=epithelium; CN=connective tissue).

FIG. 6 shows the morphology of porcine buccal tissue (EP=epithelium; CN=connective tissue) after passive permeation of 0.5% ODAN.HCl.

FIG. 7 shows the morphology of porcine buccal tissue (EP=epithelium; CN=connective tissue) after iontophoresis 0.3 mA for 8 hours.

FIG. 8 shows the morphology of porcine buccal tissue (EP=epithelium; CN=connective tissue) after combined treatment of iontophoresis 0.3 mA for 8 hours+5% DDAIP.HCl in water 1 hour pretreatment.

FIG. 9 shows the morphology of porcine buccal tissue (EP=epithelium; CN=connective tissue) after combined treatment of iontophoresis 0.3 mA for 8 hours+5% DDAIP.HCl in PG 1 hour pretreatment.

FIG. 10 shows the morphology of porcine buccal tissue (EP=epithelium; CN=connective tissue; white area, damaged area) after combined treatment of iontophoresis 0.3 mA for 8 hours+10% oleic acid in PG 1 hour pretreatment.

FIG. 11 is a graphic representation of the EpiOral™ tissue viability (%) of different treatments for 4 hours with data presented as means±S.D. (N=2).

FIG. 12 is a graphic representation of the Exposure Time (ET) value of 5% DDAIP.HCl in water in a dose response curve from EpiOral™ tissue (N=2).

FIG. 13 is a graphic representation of the enhancement ratios (ER) of iontophoresis on transdermal and transbuccal delivery of lidocaine HCl at 8 hours, with data presented as means±S.D. (3≦N≦9).

FIG. 14 is a graphic representation of the enhancement ratios (ER) of iontophoresis on transdermal and transbuccal delivery of nicotine hydrogen tartrate at 8 hours, with data presented as means±S.D. (3≦N≦9).

FIG. 15 is a graphic representation of the enhancement ratios (ER) of iontophoresis on transdermal and transbuccal delivery of diltiazem HCl at 8 hours, with data presented as means±S.D. (3≦N≦9).

FIG. 16 is a graphic representation of the enhancement ratios (ER) of enhancers on transdermal and transbuccal delivery of lidocaine HCl at 8 hours, with data presented as means±S.D. (3≦N≦9).

FIG. 17 is a graphic representation of the enhancement ratios (ER) of enhancers on transdermal and transbuccal delivery of nicotine hydrogen tartrate at 8 hours, with data presented as means±S.D. (3≦N≦9).

FIG. 18 is a graphic representation of the enhancement ratios (ER) of enhancers on transdermal and transbuccal delivery of diltiazem HCl at 8 hours, with data presented as means±S.D. (3≦N≦9).

FIG. 19 is a graphic representation of the enhancement ratios (ER) of enhancers on transdermal and transbuccal delivery of lidocaine HCl at 8 hours, with data presented as means±S.D. (3≦N≦9).

FIG. 20 is a graphic representation of the enhancement ratios (ER) of combined iontophoresis and enhancers on transdermal and transbuccal delivery of nicotine hydrogen tartrate at 8 hours, with data presented as means±S.D. (3≦N≦9).

FIG. 21 is a graphic representation of the enhancement ratios (ER) of combined iontophoresis and enhancers on transdermal and transbuccal delivery of diltiazem HCl at 8 hours, with data presented as means±S.D. (3≦N≦9).

### DESCRIPTION OF PREFERRED EMBODIMENTS

The term “buccal” and “oral composition” as used herein and in the appended claims denotes administering an active therapeutic agent from a matrix comprising an alkyl N,N-disubstituted amino acetate in said matrix to the oral mouth cavity of a subject. The oral composition is preferably in the form of a gel, orally disintegrating tablet (for buccal or sublingual use). A preferred buccal delivery system comprises a matrix for containing and releasing the therapeutic agent into the oral cavity and an alkyl N,N-disubstituted amino acetate in said matrix as a drug-releasing enhancement agent. The buccal delivery system preferably is a gel, a patch or a tablet.

The term “therapeutic agent,” as used herein and in the appended claims denotes a compound, including a protein or a peptide, that has active therapeutic, phamacokinetic properties and utility. Illustrative categories of therapeutic agents suitable for practicing the present invention are anesthetics, antihistamines, antipsychotics, acetylcholinesterase inhibitors, analgesics, benzodiazepines, antipyretics, anticonvulsants, triptans/serotonin agonists, non-steroidal anti-inflammatory drugs (NSAIDS), antiemetics, corticosteroids, DDC inhibitors, proton pump inhibitors, antidepressants, anticholinergics, monoamine oxidase inhibitors (MAOIs), dopamine receptor antagonists, nonbenzodiazepine hypnotics, narcotics, nicotine replacement therapy agents, hormones, oral fungicides, opioid analgesics, small molecule therapeutics, vasodilators, vasoconstrictors, and the like.

As used herein, the term “physiologically acceptable carrier” refers to a diluent, adjuvant, excipient, or the like tablet vehicle in which a therapeutic agent is administered. Such carriers can include starch, glucose, lactose, sucrose, gelatin, malt, rice, flour, chalk, silica gel, sodium stearate, glycerol monostearate, talc, sodium chloride, dried skim milk, or any compound found in the Handbook of Pharmaceutical Excipients (4^{th} edition, Pharmaceutical Press) and the like. A minor amount of wetting or emulsifying agents, or pH buffering agents such as acetates, citrates, or phosphates may also be present. Also, antibacterial agents such as methyl parabens; antioxidants such as ascorbic acid or sodium bisulfite; chelating agents such as ethylenediaminetetraacetic acid; and agents for the adjustment of tonicity such as sodium chloride or dextrose may be present.

The term “therapeutically effective amount” refers to those amounts that, when administered to a particular subject in view of the nature and severity of that subject's disease or condition, will have a desired therapeutic effect, e.g., an amount which will cure, prevent, inhibit, or at least partially arrest or partially prevent a target disease or condition.

Embodiments of alkyl N,N-disubstituted amino acetates suitable for present purposes are represented by the formula:

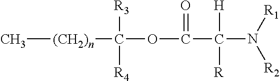

wherein n is an integer having a value in the range of about 4 to about 18; R is a member of the group consisting of hydrogen, C₁ to C₇ alkyl, benzyl and phenyl; R₁ and R₂ are members of the group consisting of hydrogen and C₁ to C₇ alkyl; and R₃ and R₄ are members of the group consisting of hydrogen, methyl and ethyl.

Preferred alkyl (N,N-disubstituted amino)-acetates are C₄ to C₁₈ alkyl (N,N-disubstituted amino) acetates and C₄ to C₁₈ alkyl (N,N-disubstituted amino) propionates as well as pharmaceutically acceptable salts and derivatives thereof. Exemplary specific alkyl 2-(N,N-disubstituted amino) acetates include dodecyl 2-(N,N dimethylamino) propionate (DDAIP);

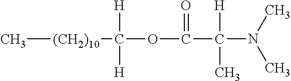

and dodecyl 2-(N,N-dimethylamino) acetate (DAA);

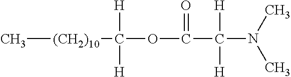

Preferred are dodecyl-2-(N,N-dimethylamino) propionate (DDAIP); dodecyl-2-(N,N-dimethylamino) acetate (DAA); 1-(N,N-dimethylamino)-2-propyl dodecanoate (DAIPD); 1-(N,N-dimethylamino)-2-propyl myristate (DAIPM); 1-(N,N-dimethylamino)-2-propyl oleate (DAIPO); and pharmaceutically acceptable acid addition salts thereof.

Particularly preferred is the hydrochloride of DDAIP (DDAIP.HCl). DDAIP.HCl is available from Steroids, Ltd. (Chicago, Ill.), Pisgah Laboratories (Pisgah Forest, N.C.), and SAI Advantium (India). The preparation of DDAIP and crystalline acid addition salts thereof is described in U.S. Pat. No. 6,118,020 to Büyüktimkin, et al., which is incorporated herein by reference. Long chain similar amino substituted, alkyl carboxylic esters can be synthesized from readily available compounds as described in U.S. Pat. No. 4,980,378 to Wong, et al., which is incorporated herein by reference to the extent that it is not inconsistent herewith.

As described therein, alkyl-2-(N,N-disubstituted amino) acetates are readily prepared via a two-step synthesis. In the first step, long chain alkyl chloroacetates are prepared by reaction of the corresponding long chain alkanols with chloromethyl chloroformate or the like in the presence of an appropriate base such as triethylamine, typically in a suitable solvent such as chloroform. The reaction can be depicted as follows:

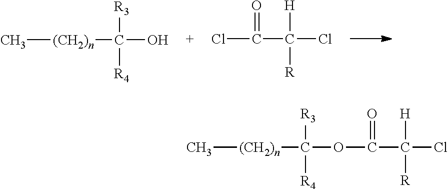

wherein n, R, R₁, R₂, R₃ and R₄ are defined as above. The reaction temperature may be selected from about 10 degrees Celsius to about 200 degrees Celsius or reflux, with room temperature being preferred. The use of a solvent is optional. If a solvent is used, a wide variety of organic solvents may be selected. Choice of a base is likewise not critical. Preferred bases include tertiary amines such as triethylamine, pyridine and the like. Reaction time generally extends from about one hour to three days.

In the second step, the long chain alkyl chloroacetate is condensed with an appropriate amine according to the scheme:

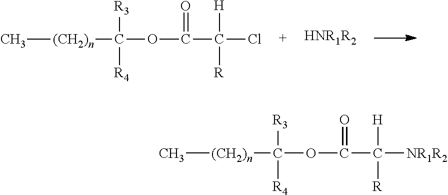

wherein n, R, R₁, R₂, R₃ and R₄ are defined as before. Excess amine reactant is typically used as the base and the reaction is conveniently conducted in a suitable solvent such as ether. This second step is preferably run at room temperature, although temperature may vary. Reaction time usually varies from about one hour to several days. Conventional purification techniques can be applied to ready the resulting ester for use in a pharmaceutical compound.

The amount of alkyl N,N-disubstituted amino acetate, such as DDAIP, present in the buccal or sublingual therapeutic compositions can vary, and depends in part on the particular therapeutic agent to be administered as well as the buccal or sublingual route of oral administration.

As used herein, the term “small molecule therapeutic” is a low molecular weight organic compound which is not a polymer but binds with relatively high affinity to a biopolymer such as a protein, a nucleic acid, or polysaccharide and also alters the activity or function of the biopolymer. The upper molecular weight limit for a small molecule therapeutic is about 1000 Daltons which allows for diffusion across all membranes so that intracellular sites of action can be reached. Very small oligomers are also considered small molecules, e.g., dinucleotides, disaccharides, and the like. Illustrative are the taxanes, mesalamine (Pentasa®), motexafin gadolinium, temozolomide, tarceva, sensipan, safinamide, simvastatin, pravastatin, sildenafil, peptide mimetics, the siRNAs, and the like. Taxanes are diterpenes utilized in cancer chemotherapy. Particularly well suited taxanes for compositions of the present invention are paclitaxel, docetaxel, and tesetaxel.

Illustrative hormones suitable for buccal administration are the insulins, e.g., human insulin, bovine insulin, porcine insulin, biosynthetic human insulin (Humulin®) etc., somatostatin, vasopressin, calcitonin, estrogen, progestin, testosterone, glucagon, glucagon-like peptide (GLP-1) and its analogs, and the like. For example, a composition comprising insulin and dodecyl 2-(N,N-dimethylamino) propionate hydrochloride is particularly well suited for controlling blood glucose levels in diabetic patients.

Examples of suitable opioid analgesics are morpine and morphine derivatives such as fentanyl and sulfentanil. Example NSAIDs include acylpropionic acid derivates, such as ibuprofen, salicylic acid derivatives, and the like. Example anticonvulsants include iamotrigine, phenobarbital, phenyloin, and the like. Example benzodiazepines include clonazepam, diltiazem, particularly diltiazem hydrochloride (DHCl), and the like. Example triptans/serotonin agonist includes rizatriptan, zolmitriptan, and the like. Example antiemetics include ondansetron, particularly ondansetron hydrochloride (ODAN.HCl), scopolamine, and the like. Example local anesthetics include lidocaine, particularly lidocaine hydrochloride (LHCl). Example nicotine replacement therapy agents include nicotine hydrogen tartrate (NHT).

Buccal administration (in the pouch of the cheek of the subject) is particularly useful for active therapeutic agents which show poor bioavailability upon administration through other non-parenteral modes. It is necessary for a buccal composition to remain in contact with the oral mucosa for a time sufficient for absorption of the medicament to be administered. If the formulation falls apart too quickly, the active ingredient is swallowed, and an insufficient amount of medicament is delivered. If the formulation does not fall apart quickly enough, patient compliance difficulties can result, since the patient should not eat or drink while using the buccal composition. The composition should be of a small size to avoid discomfort to the patient and it is desirable that as much of the composition as possible be soluble in saliva so that discomfort in the form of insoluble grit or components in the mouth can be avoided.

Patches are a convenient form for transbuccal delivery and comprise a reservoir or matrix that contains the therapeutic drug designed to be released at a constant rate over a period of several hours to days after placement of the patch in contact with the buccal tissue. A “general” patch typically consists of a release liner which protects the patch during storage and which is removed prior to use; a drug solution or gel in direct contact with the release liner; pressure sensitive adhesive that provides adherence to the skin and may also be the matrix in which the drug may be incorporated; a backing laminate that protects the patch from the environment; and optionally, a rate controlling membrane that regulates the release of the drug from the reservoir.

Typically, there are four main types of patches such as the following. 1) Single-layer Drug-in-Adhesive type in which the drug is included directly within the skin/buccal contacting adhesive. In this type of patch the adhesive layer acts as a drug reservoir and releases the active drug into the skin/buccal membrane as well as adhering the patch to the tissue. The adhesive layer is sandwiched by a temporary liner and a removable backing. 2) Multi-layer Drug-in-Adhesive type in which the drug is incorporated directly into the adhesive, and adds another layer of drug-in-adhesive, usually separated by a membrane. This patch is also sandwiched by a temporary liner-layer and a permanent backing. 3) Reservoir type system includes a liquid compartment containing drug solution or suspension or gel separated from the release liner by a semi-permeable membrane and adhesive. The adhesive component can either be a continuous layer between the membrane and the release liner or as a concentric configuration around the membrane. 4) Matrix type system which has a drug layer of a semisolid matrix containing a drug solution or suspension or gel which is in direct contact with the release liner and the adhesive layer which is attached to the backing layer. Matrix patches optionally have a rate controlling membrane. Matrix patch systems are presently preferred.

An example of an orally disintegrated tablet composition for buccal administration of an active therapeutic agent comprises (a) about 1 to about 20% by weight of a soluble, pharmaceutically acceptable polymeric adhesive; (b) about 1 to about 10% by weight of a pharmaceutically acceptable tablet disintegrant; (c) a soluble, directly compressible tablet excipient; (d) a therapeutically useful amount of active therapeutic agent; and (e) an alkyl N,N-disubstituted amino acetate.

The soluble, pharmaceutically acceptable polymeric adhesive is useful to provide tackiness to the buccal formulation so that it will be held in place upon administration. The amount of adhesive in the formulation is about 1-20% by weight, preferably about 2-10%. Use of amounts less than 1% may result in insufficient adhesive properties or the formulation falling apart too quickly, while excessive amounts may result in the formulation lasting for a longer period than is desirable. The adhesives desirably are sticky when moist, but not when dry, for convenience in handling. The amount of adhesive which can be used increases with the solubility of the active ingredient.

One particularly desirable group of polymeric adhesives for oral use are high molecular weight polymers of acrylic acid known as carbomers. Molecular weights of 450,000 to 4,000,000 are useful, with a molecular weight of about 3,000,000 (carbomer 934 P) being preferred. These substances are sold by B. F. Goodrich under the trademark Carbopol®. The adhesives have been found to allow use of minimal amounts to provide the desired adhesive characteristics to the formulation, which is advantageous since increasing amounts of adhesive may impede the dissolution of the active ingredient. Other suitable hydrophilic polymers include partially (87-89%, for example) hydrolyzed polyvinylalcohol (molecular weight 10,000 to 125,000, preferably 11,000 to 31,000), polyethylene oxide (molecular weight about 100,000 to about 5,000,000, preferably 400,000) and polyacrylates, such as that sold by GAF under the trademark Gantrez®, particularly those designated as high molecular weight polyacrylates. Hydroxypropyl methylcellulose, having a molecular weight of 13,000 to 140,000 (sold under the trademark Methocel® by Dow), and hydroxypropyl cellulose, having a molecular weight of 60,000 to 1,000,000 (sold under the trademark Klucel®) also are useful adhesives. Material toward the high end of each of the molecular weight ranges are preferred. The term “soluble” is used as an indication that the material is soluble in water or saliva. Upon administration, the adhesive forms a gel-like substance which is gradually broken up by a pharmaceutically acceptable disintegrant which swells upon administration, thus exposing more of the formulation to saliva. This causes the preparation to break up gradually.

The amount of disintegrant in a tablet formulation is about 1 to 10% by weight, preferably 3-6%. Excessive amounts of disintegrant actually may unduly delay disintegration, as by formulation of an insoluble gel, instead of aiding dissolution of the formulation by expansion. One useful disintegrant is the material crospovidone, which is a cross-linked polyvinylpyrrolidone product. This material is sold under the trademark Polyplasdone XL by GAF. Other useful disintegrants include Ac-di-sol® (FMC's trademark for croscarmellose, a cross-linked carboxylic methylcellulose), alginic acid, sodium carboxymethyl starch such as that sold as Explotab® by Edward Mendell Co., Inc., starch, calcium carboxymethyl cellulose, sodium starch glycolate, microcrystalline cellulose, and the like.

A tablet formulation can also include a soluble, directly compressible tableting excipient such as a sugar. One such useful tableting excipient is a co-crystallization of 97% sucrose-3% highly modified dextrins sold under the trademark Di-Pac® by Amstar. Other such excipients known to those skilled in the art, such as lactone, spray-dried lactose, and the like also may be used. The amount of excipient used is such that the resulting formulation is big enough to be handled conveniently, yet small enough to dissolve properly. Other tablet ingredients which may be used include lubricants such as magnesium stearate in the amount of up to about 1% by weight, preferably 0.5%, and coloring or flavoring agents.

Tablet formulations of the present invention can be prepared by mixing the ingredients together and compressing desired amounts of the mixture into tablet form. The final products for buccal or sublingual administration desirably have a diameter of about a quarter inch (0.635 cm) and a thickness of about 0.05 inches (0.127 cm), and upon administration disintegrate over a period of about 30 seconds to 20 minutes, preferably about 2-12 minutes.

Preferably the matrix for a buccal delivery system in the form of a tablet includes, in addition to the alkyl (N,N-disubstituted amino) acetate, a hydrophilic polymeric material, such as a crosslinked hydrophilic polymer, to allow swelling of the matrix, but not dissolution into the oral cavity. The matrix polymer is chosen based on the molecular weight, hydrophobicity or hydrophilicity of the therapeutic agent and the desired release rate. Thus, for delivery of a hydrophilic therapeutic agent, a suitable polymer would be a lightly crosslinked hydrogel which would allow for water absorption and swelling permitting the hydrophilic therapeutic agent to be released in the oral cavity. The degree of the polymer hydrophobicity/hydrophilicity will dictate the rate of release and the duration of activity.

An example compressible matrix for a buccal delivery system comprises: (a) a physiologically acceptable carrier, such as a soluble, pharmaceutically acceptable polymeric adhesive, a pharmaceutically acceptable tablet disintegrant, and a soluble, directly compressible tablet excipient; and (b) an alkyl N,N-disubstituted amino acetate. The matrix can be prepared as an article of manufacture, and stored as an uncompressed mixture until the therapeutic agent of choice is to be added and after which the final formulation is then compressed.

The active therapeutic agents useful with this invention include those mentioned above. Of course, the amount will vary depending upon the dosage desired for a given treatment.

The foregoing description and the following examples are intended as illustrative but are not to be taken as limiting. Still other variations within the spirit and scope of the present invention are possible, and will readily present themselves to those skilled in the art.

### Materials and Methods

### I. Therapeutic Agents

A. Diltiazem hydrochloride (DHCl) (Dilacor XR®, Watson) is a calcium ion influx inhibitor (slow channel blocker or calcium antagonist). Diltiazem hydrochloride is a 1,5-Benzothiazepin-4(5H) one, 3-(acetyloxy)-5-[2-(dimethylamino)ethyl]-2,3-dihyro-2-(4-methoxyphenyl)-, monohydrochloride, (+)-cis-. The molecular formula of DHCl is C₂₂H₂₆N₂O₄S.HCl and its molecular weight is 450.98. Dilacor XR® is indicated for the treatment of hypertension. Diltiazem hydrochloride may be used alone or in combination with other antihypertensive medications, such as diuretics. Dilacor XR® is also indicated for the management of chronic stable angina. Diltiazem hydrochloride is a white to off-white crystalline powder with a bitter taste. It is soluble in water, methanol, and chloroform and light sensitive. Dilacor XR® capsules have different dosage strengths such as 120 mg, 180 mg, or 240 mg that allows for the controlled release of DHCl over a 24-hour period. DHCl dihydrate was obtained from Polymed, Inc. (Houston, Tex.).

B. Ondansetron hydrochloride (ODAN.HCl) is a selective blocking agent of the serotonin 5-HT3 receptor that is used to prevent post-operative nausea and vomiting (antiemetic). It is the active ingredient in ZOFRAN® Orally Disintegrating Tablets (Glaxo Wellcome SmithKline) as the dihydrate, the racemic form of ondansetron-(±) 1,2,3,9-tetrahydro-9-methyl-3-[(2-methyl-1H-imidazol-1-yl) methyl]-4H-carbazol-4-one, monohydrochloride, dihydrate. The empirical formula of ODAN.HCL is C₁₈H₁₉N₃O.HCl.2H₂O with a molecular weight of 365.9. ODAN.HCl dihydrate was obtained from Polymed, Inc., (Houston, Tex.). While the tablet or injectable dosage form of ODAN.HCl is clinically proven to be effective, patients have to endure either painful injection or the side effects associated with gastrointestinal (GI) absorption. Therefore, it is desirable to develop an alternative approach to promoting patients' compliance and reduce the effects of GI absorption and the issues with oral administration with accompanying nausea and vomiting.

C. Lidocaine hydrochloride (LHCl) is a local anesthetic, chemically designated as 2-(Diethylamino)-2′,6′-acetoxylidide mono-hydrochloride, monohydrate, is a white crystalline powder freely soluble in water. The empirical formula is C₁₄H₂₂N₂O.HCl with a molecular weight of 288.81, pKa=7.8. LHCl was obtained from Sigma Aldrich (Saint Louis, Mo.).

D. Nicotine hydrogen tartrate (NHT), a nicotine replacement, has a molecular weight of 462, is a white powder and is soluble in water. Every 3 grams of nicotine hydrogen tartrate is equivalent to about 1 gram of Nicotine-(1-methyl-2(3-pyridyl) pyrrolidine. NHT was obtained from Sigma Aldrich (Saint Louis, Mo.). The therapeutic indication for NHT includes restraining the desire for cigarette smoking and eliminating the addiction gradually through delivering small and controlled doses of nicotine into the bloodstream without consuming other toxic and dangerous chemicals present in cigarette smoke.

### II. Materials

Dodecyl-2-N,N-dimethylaminopropionate (DDAIP) and dodecyl-2-N,N-dimethylaminopropionate hydrochloride (DDAIP.HCl) were provided by NexMed (San Diego, Calif.).

Azone and Br-iminosulfurane were synthesized at New Jersey Center for Biomaterials, Rutgers—The State University of New Jersey, (Piscataway, N.J.).

Porcine buccal tissue was obtained from Barton's Farms and Biologicals (Great Meadow, N.J.).

Silver wire, propylene glycol (PG) (ReagentPlus®, 99%) and citric acid were purchased from Sigma Aldrich, (Saint Louis, Mo.).

Phosphate buffer saline tablets were purchased from MP Biomedicals, LLC (Solon, Ohio).

Cellulose gum (CMC) was provided by TIC Gums (Belcamp, Md.).

Tissue-Tek® compound was purchased from Sakura Finetek USA, Inc., (Torrance, Calif.). Formalin 10% was purchased from Fisher Scientific.

MTS-CeliTiter 96® AQueous One Solution Reagent was purchased from Promega Corp., (Madison, Wis.).

DMEM and EpiLife® mediums were purchased from Invitrogen Corp., (Carlsbad, Calif.).

Gelva® GMS 3083 adhesive—ethyl acetate was provided by CYTEC Products, Inc., (Elizabethtown, Ky.).

3M Scotchpak™ 9732 Backing—polyester film laminate and 3M Scotchpak™ 9741 Release Liner—fluoropolymer coated polypropylene film were provided by 3M, Inc., (St. Paul, Minn.).

EpiOral™ Tissue (ORL-202) was purchased from MatTek Corporation (Ashland, Mass.).

Nikon Eclipse E 800 light microscope and Nikon Digital Camera (Model DXM 1200) (Micro Optics, Cedar Knolls, N.J.) were used for all histological studies.

HPLC System (Model: Agilent or HP 1100 series).

### III. Methodology

### Buccal Tissue Preparation

Buccal mucosa samples with underlying connective tissue were surgically removed from the pig check area and stored under −30° C. for future use. Prior to use, the samples were thawed at room temperature for at least 3 hours. Then the underlying connective tissue was removed using a scalpel blade and the remaining buccal mucosa was then carefully trimmed using surgical scissors to a thickness of about 300-400 μm. The buccal tissues were placed in phosphate buffered saline (PBS) with pH 7.5 for 1 hour prior to use.

### IV. Equipment and Methodology

Franz diffusion cells (PermeGear, Hellertown, Pa.) were used for all in vitro permeation studies using buccal tissue under varying conditions: passive (control); 1 hour enhancer pretreatment, 8 hours iontophoresis (0.1, 0.2 and 0.3 mA); and combined treatment of 1 hour enhancer treatment and 8 hours iontophoresis at 0.3 mA, and then passive only up to 24 hrs. All permeation studies were performed at 37° C.

For passive permeation studies, the Franz cell receptor compartment was filled with PBS solution and stirred at 600 rpm. The buccal tissue was placed in between the donor and receptor compartments with the side of connective tissue facing the donor compartment. The available diffusion area was 0.64 cm². A volume 0.3 ml of the drug formulation was added into the donor compartment at the beginning of the experiment. At different time points (0.0, 0.5, 1. 3, 5, 8, 12, 20, 24 hrs), 300 μl sample was withdrawn from receptor compartment for HPLC analysis and immediately replaced with 300 μl of PBS (pH=7.5).

For enhancer pretreatment studies, the same procedures described above for passive permeation were followed except that the buccal tissue was pretreated for 1 hr by adding 30 μl of chemical enhancer solution on top of buccal tissue in the donor compartment prior to the application of 0.3 ml drug formulation.

For iontophoresis, a Phoresor II Auto—Iontophoresis Power Device (Model PM 850) Iomed, Inc., provided 0.1, 0.2 and 0.3 MA for 8 hrs of treatment. The anodal electrode (Ag) was placed in the gel formulation in the donor compartment about 2 mm above the buccal tissue membrane. The cathode (AgCl) was inserted into the receptor compartment. After 8 hours, iontophoresis was discontinued and then the passive-only permeation continued for 16 hrs. The sampling method and time points were the same as for passive and chemical enhancer pretreatment experiments.

An 8 hour iontophoresis period is referred to herein as Stage I. A post −8 hour iontophoresis period, passive only permeation period is referred to herein as Stage II.

### Preparation of Ag and Ag Cl Electrodes

Pure silver (Ag) wire with 0.5 mm in diameter was used as the anodal electrode. An AgCl electrode was prepared by dipping silver chloride powder coated silver wire and a pure silver wire into 0.1 N HCl solution, and connecting them to a power source 3 mA for 12 hours. The purple layer coated silver wire—AgCl electrode was used as a cathodal electrode in the iontophoretic studies.

### V. Data Analyses

The steady state flux at time t (J·μg cm⁻²) was calculated from the slope of the linear portion of the profile of cumulative drug amounts permeated vs. time. The cumulative drug amount in the receptor compartment after 8 hrs and 24 hrs was defined as Q₈ and Q₂₄ (μg cm⁻²), respectively. The enhancement ratio (ER) for flux was calculated as follows:

$$\frac{\begin{matrix}ER = Flux for treated buccal tissue with enhancer \\ or iontophoresis or their combination\end{matrix}}{flux for untreated buccal tissue}$$

Results were presented as mean±standard error (S.D.) (n) where n represented the number of replicates. Data analysis of ER was performed for treated tissue against control by the unpaired Student's t-test. ANOVA was used to compare ER fluxes among different treated tissues. A probability of less than 5% (p<0.05) was considered significant.

### Example 1

### Drug Gel Composition with Diltiazem HCl (DHCl)

Gel drug compositions containing 3% DHCl with DDAIP enhancer were prepared as follows with all amounts in w/v, final composition basis.

Composition A. 3% DHCl with 5% DDAIP.HCl in a 4% HPMC aqueous gel.

Hydroxylpropyl methylcellulose (HPMC) (4%) (Methocel K₁₅M premium grade—HPMC, Dow Chemicals, Inc., Auburn Hills, Mich.) was uniformly dispersed in deionized water (88%) to form a clear gel. DDAIP.HCl (5%) was dispersed into the HPMC gel and mixed until uniform. Then DHCl HCl (3%) was added into the gel and mixed until uniform using a lightning mixer to form a 3% DHCl composition with 5% DDAIP.HCl enhancer in the final gel composition (pH 5.5; viscosity (RV/E/2 min) 400,000 cps).

Composition B. 3% DHCl with 5% DDAIP.HCl in a 4% HPMC aqueous gel.

The procedure of Composition A was repeated, except that DDAIP (5%) was the enhancer dispersed into the HPMC gel and mixed until uniform. Then DHCl (3%) was added into the gel andmixed until uniform using a lightning mixer to form a 3% DHCl gel composition with 5% DDAIP enhancer in the final gel composition (pH 5.8; viscosity (RV/E/2 min) 400,000 cps).

Composition C (Comparative). 3% DHCl in a 4% HPMC aqueous gel.

Hydroxylpropyl methylcellulose (HPMC) (4%) was uniformly dispersed in deionized water to form a clear gel. Then DHCl (3%) was added into the HPMC gel and mixed until uniform using a lightning mixer to form an aqueous 3% DHCl gel (pH=6.0; viscosity (RV/E/2 min)=400,000 cps).

### Example 2

### Drug Gel Composition with Ondansetron HCl (ODAN.HCl)

Gel drug compositions containing 2% ODAN.HCl with DDAIP enhancer were prepared as follows with all amounts in w/v, final composition basis.

Composition A. 2% ODAN.HCl with 5% DDAIP.HCl in a 4% HPMC aqueous gel.

Citric acid (0.02%) was dissolved in deionized water (88.98%) and then hydroxylpropyl methylcellulose (HPMC) (4%) (Methocel K15M premium grade—HPMC, Dow Chemicals, Inc., Auburn Hills, Mich.) was added and mixed well to form a uniform clear gel. DDAIP.HCl (5%) was dispersed into the HPMC gel and mixed until uniform. Then ODAN HCl (2%) was added into the gel and mixed until uniform using a lightning mixer to form a 2% ODAN.HCl gel composition with 5% DDAIP.HCl in the fluid composition (pH 3.6; viscosity (RV/E/2 min) 500,000 cps).

Composition B. 2% ODAN.HCl with 5% DDAIP in a 4% HPMC aqueous gel.

The procedure of Composition A was repeated, except that DDAIP (5%) was the enhancer dispersed into the HPMC gel and mixed until uniform. Then ODAN.HCl (2%) was added to the gel and mixed until uniform using a lightning mixer to form a 2% ODAN.HCl composition with 5% DDAIP.HCl in the final gel composition (pH 3.8; viscosity (RV/E/2 min) 500,000 cps).

Composition C (Comparative). 3% ODAN.HCl in an aqueous 4% HPMC gel.

Citric acid (0.02%) was dissolved in deionized water (93.98%) and then hydroxylpropyl methylcellulose (HPMC) (4%) was added and mixed well to form a uniform clear gel. ODAN.HCl (2%) was added into the gel and mixed until uniform using a lightning mixer to form an aqueous 2% ODAN.HCl gel (pH 3.6; viscosity (RV/E/2 min) 500,000 cps).

### Example 3

### Drug Patch with Diltiazem HCl (DHCl)

Matrix type transbuccal patches having a drug layer of semisolid matrix containing a drug gel, which is in direct contact with the release liner, with the adhesive layer attached to the backing layer, were prepared. Patches were separately prepared with the gel compositions A, and C of Example 1 as follows:

Step (a) Prepare a 3% DHCl gel as described in Example 1, Composition A. Prepare a drug patch containing the composition of Example 1A by the following steps.

Step (b) Preparation of adhesive and backing layer: add 10 grams of adhesive (Ethyl acetate, GMS3080 from Cytec Gelva (Springfield, Mass.)) to a 20×30 cm² of backing laminate roll (3M Scotchpak™ 9732 Backing Polyester Film Laminate, Saint Paul, Minn.), then use a Drawdown machine (lab scale, Accu-lab™ JR from Industry Tech., Inc., Oldsmar, Fla.) to roll on the adhesive on the backing laminate roll to form a thickness of 0.058″ uniformed layer of adhesive on the backing laminate.

Step (c) Patch completion: About 0.3 ml of a 3% DHCl gel composition of Example 1A from Step 1 was added uniformly on the side of adhesive layer attached on backing laminate (1×1 cm²), then a release liner (2×2 cm²) (Fluoropolymer Coated Polyester Film, 3M Scotchpak™ 1020) was placed on top of the DHCl gel from Step 1. Finally, a configuration 1×1 cm² of 3% DHCl patch was obtained by using a punching machine (F-2000 MB Cartoning machine, Bloomington, Minn.) to punch through the DHCl gel sandwiched by backing laminate and release liner.

A comparative drug patch was prepared by repeating the above procedure, except that the gel composition of Example 1C was used.

### Example 4

### Drug Patch Formulation with Ondansetron HCl (ODAN.HCl)

A matrix type of transbuccal patch was prepared following the steps of (b) and (c) of the procedure of EXAMPLE 3, except that the gel composition A of Example 2 and comparative gel composition C of Example 2 were used.

### Example 5

### Transbuccal Delivery Systems

In vitro passive transbuccal delivery permeation studies of several 3% DHCl and 2% ODAN HCl patches of EXAMPLES 3 and 4, respectively, were performed using a Franz cell diffusion model using porcine buccal mucosa tissues. Enhancement effects of iontophoresis (0.3 mA for 8 h) were also evaluated on transbuccal delivery of 3% DHCl and 2% ODAN.HCl patches with and without DDAIP.HCl enhancer (comparative patch Examples 3C and 4C) and on comparative gel compositions of Examples 1C and 2C. The methodology for passive permeation, iontophoresis and data analyses is described above in the materials and methods sections III, IV and V for transbuccal permeation studies.

I. In Vitro Transbuccal Permeation Study—DHCl

The flux and calculated enhancement ratio (ER) for HHCl transbuccal permeation is shown in Tables 1 and 2.

Tables 1 and 2 show that the comparative 3% DHCl patch of Example 3C provided skin with exclusivity which possibly resulted in higher transbuccal permeation than comparative 3% DHCl gel of Example 1C. When compared to passive patch permeation, both iontophoresis (0.3 mA for 8 h) (Stage I) and 5% DDAIP.HCl provided significantly higher permeability of DHCl via porcine buccal tissue during a 24 h study period. It was noted that 5% DDAIP.HCl patch of Example 3B provided a greater enhancement effect than iontophoresis (0.3 mA for 8 h) during the entire 24 h period of the study. It was noted that the enhancement effect from post-iontophoresis (Stage II) was not significantly reduced after iontophoresis was discontinued at 8 h (Stage I), indicating that an iontophoretic enhancement effect was not primarily electrorepulsion driven and contribution from electroosmosis may be significant as well.

In summary, the 3% DHCl patch formulation delivered a greater amount of DHCl through porcine buccal tissue when compared to the gel formulation at the same drug concentration. It was noted that DDAIP.HCl treatment alone provided a greater enhancement effect than iontophoresis alone. It was noted that transbuccal route of DHCl delivery using patch formulations was an effective delivery route.

TABLE 1
3% DHCl Transbuccal Permeation Study (0-8 h) (Stage I^{a})

| | Flux | Q₈ | |
|---|---|---|---|
| Formulation | (μg/cm²*h) | (μg/cm²) | ER |
| Gel | 24.1 ± 8.16 | 170.3 ± 58.20 | 1.0 |
| Patch | 41.0 ± 3.8^{b} | 290.8 ± 42.3 | 1.7 |
| Patch + 0.3 mA | 160.1 ± 100.3^{b, c} | 1344.5 ± 611.4 | 6.6 |
| Patch + 5% | 185.9 ± 101.1^{b, c} | 1212.5 ± 911.7 | 7.7 |
| DDAIP•HCl | | | |
| Patch + 5% | 266.4 ± 59.5^{b, c} | 1945.0 ± 642.3 | 11.1 |
| DDAIP•HCl + | | | |
| 0.3 mA | | | |

^{a}Data are presented as means ± S.D. (3 ≦ N ≦ 8).
^{b}Statistically significantly higher than gel (p < 0.05) (Student's t-test).
^{c} Statistically significantly higher than patch and gel (p < 0.05) (ANOVA).

TABLE 2
3% DHCl Transbuccal Permeation Study (8-24 h) (Stage IP)

| | Flux | Q₂₄ | |
|---|---|---|---|
| Formulation | (μg/cm²*h) | (μg/cm²) | ER |
| Gel | 51.7 ± 11.5 | 941.2 ± 210.0 | 1.0 |
| Patch | 61.4 ± 4.3 | 1230.8 ± 63.6 | 1.2 |
| Patch + 0.3 mA | 143.9 ± 52.3^{b} | 3535.1 ± 1704.9 | 2.8 |
| Patch + 5% | 208.6 ± 17.1^{b} | 4634.2 ± 1186.0 | 4.0 |
| DDAIP•HCl | | | |
| Patch + 5% | 176.4 ± 13.2^{b} | 5149.4 ± 608.23 | 3.4 |
| DDAIP•HCl + | | | |
| 0.3 mA | | | |

^{a}Data are presented as means ± S.D. (3 ≦ N ≦ 8).
^{b}Statistically significantly higher than patch and gel (p < 0.05) (ANOVA).

II. In Vitro Transbuccal Permeation Study—ODAN.HCl

The flux and calculated enhancement ratio (ER) for ODAN.HCl transbuccal permeation is shown in Tables 3 and 4.

Tables 3 and 4 show that the cumulative amount permeated from a 2% ODAN HCl patch of Example 4A was comparable to 2% ODAN.HCl gel of Example 2C during the 24 h period of study. When compared to passive patch permeation, both iontophoresis (0.3 mA for 8 h) (Stage I) and 5% DDAIP.HCl patch of Example 4A provided significantly higher permeability of ODAN.HCl via porcine buccal tissue during a Stage II 24 h study period. It was observed that 5% DDAIP.HCl provided a greater enhancement effect than iontophoresis (0.3 mA for 8 h) during the entire 24 h period of study. It was also noted that enhancement effect from post-iontophoresis (Stage II) was not significantly reduced after iontophoresis was discontinued at 8 h (Stage I), indicating that the iontophoretic enhancement effect was not primarily electrorepulsion driven and contribution from electroosmosis may be significant as well.

Iontophoresis (0.3 mA for 8 h) significantly enhanced transbuccal delivery of ODAN.HCl in gel and patch delivery systems. In the case of transbuccal delivery of ODAN.HCl, electroosmosis is believed to be important. ODAN.HCl patch with DDAIP.HCl enhancer provided significantly higher transbuccal delivery of ODAN.HCl when compared to ODAN.HCl patch and iontophoresis treatment. There were no synergistic enhancement effects observed from combined treatment of enhancer (DDAIP or DDAIP.HCl) and iontophoresis for transbuccal delivery of ODAN.HCl. However, it was noted that DDAIP.HCl treatment alone provided a greater enhancement effect than iontophoresis alone.

TABLE 3
2% ODAN•HCl Transbuccal Permeation Study (0-8 h) (Stage I)

| | Flux | Q₈ | |
|---|---|---|---|
| Formulation | (μg/cm²*h) | (μg/cm²) | ER |
| Gel | 10.3 ± 2.7 | 67.4 ± 20.9 | 1.0 |
| Patch | 9.7 ± 2.1 | 71.2 ± 22.1 | 1.0 |
| Patch + 0.3 mA | 34.9 ± 13.2^{b} | 296.6 ± 90.8 | 3.4 |
| Patch + 5% | 144.0 ± 30.6^{b, c} | 1153.6 ± 383.5 | 14.0 |
| DDAIP•HCl | | | |
| Patch + 5% | 129.3 ± 36.6^{b, c} | 1059.2 ± 441.1 | 12.6 |
| DDAIP•HCl + | | | |
| 0.3 mA | | | |

^{a}Data are presented as means ± S.D. (3 ≦ N ≦ 8).
^{b}Statistically significantly higher than gel and patch (p < 0.05) (ANOVA).
^{c} Statistically significantly higher than patch, gel and patch + 0.3 mA (p < 0.05) (ANOVA).

TABLE 4
2% ODAN•HCl Transbuccal Permeation Study (8-24 h) (Stage II^{a})

| | Flux | Q₂₄ | |
|---|---|---|---|
| Formulation | (μg/cm²*h) | (μg/cm²) | ER |
| Gel | 15.8 ± 3.9 | 310.1 ± 75.2 | 1.0 |
| Patch | 17.6 ± 2.1 | 330.5 ± 52.4 | 1.1 |
| Patch + 0.3 mA | 30.9 ± 13.7^{b} | 756.4 ± 310.4 | 2.0 |
| Patch + 5% | 43.3 ± 25.8^{b} | 2048.8 ± 130.1 | 2.7 |
| DDAIP•HCl | | | |
| Patch + 5% | 44.2 ± 24.7^{b} | 1982.2 ± 116.2 | 2.8 |
| DDAIP•HCl + | | | |
| 0.3 mA | | | |

^{a}Data are presented as means ± S.D. (3 ≦ N ≦ 8).
^{b}Statistically significantly higher than gel and patch (p < 0.05) (ANOVA).

Patch delivery systems are a feasible dosage form for the delivery of DHCl and ODAN.HCl transbuccally. DDAIP.HCl was more effective in enhancing transbuccal delivery of DHCl and ODAN.HCl in patch formulations. Overall, the transbuccal route was an effective delivery route for DHCl and ODAN.HCl in patch formulations.

### Example 6

### Transbuccal Delivery of ODAN.HCl

In this study, iontophoresis and chemical enhancers were evaluated separately as well as in combination in order to evaluate and promote transbuccal delivery of ODAN.HCl. The porcine epithelium of buccal tissue is similar to human and is non-keratinized and contains both neutral and polar lipids which are the major barriers to permeation. The chemical enhancers: DDAIP and its HCl salt DDAIP.HCl, and Br-iminosulfurane were evaluated for their abilities to enhance transbuccal delivery of ODAN.HCl with and without the use of iontophoresis.

1-Dodecylazacycloheptan-2-one (Azone), a derivative of caprolactam was used as a control enhancer. Azone is a hydrophobic substance specifically developed as a skin penetration enhancer and has been used to promote the oral mucosal absorption of salicyclic acid.

Amino acid alanine based DDAIP and its HCl salt DDAIP.HCl have low toxicity profiles and are biodegradable. These compounds were previously reported to effectively enhance the transdermal delivery of alprostadil, ketoprofen, ondansetron, miconazole, indomethacin, clonidine and hydrocortisone.

Biodegradable Br-iminosulfurane, is a low toxic aromatic S,S-dimethyliminosulfurane derivative is reportedly an effective enhancer for transdermal delivery of hydrocortisone.

However, the effects of these enhancers have not been studied for transbuccal drug delivery.

The materials and methods used are described above in the materials and methods section.

An ondansatron HCl gel was prepared as follows:

Nonionized cellulose gum (CMC) 1% (w/v) was uniformly dispersed in deionized water to obtain a gel. Then 0.5% (w/v) ODAN.HCl was added into CMC gel together with 0.01% citric acid to form a 0.5% ODAN.HCl gel.

Buccal mucosa samples were prepared as described above in the Materials and Methods Section.

Enhancer Solution Preparation

All enhancer solutions were prepared at 5% w/v or 2.5% w/v. The DDAIP.HCl solutions were prepared in either water or propylene glycol (PG). The Br-iminosulfurane, DDAIP and Azone solutions were prepared in PG only due to their low aqueous solubilities.

In Vitro Transbuccal Permeation Study

Franz diffusion cells were used for all in vitro permeation studies using buccal tissue under varying conditions: passive (control), 1 hour enhancer pretreatment, 8 hrs. iontophoresis (0.1, 0.2 and 0.3 mA), and combined treatment of 1 hr. enhancer pretreatment and 8 hrs. iontophoresis at 0.3 mA, and then passive only up to 24 hrs. All permeation studies performed at 37° C. The procedure for passive permeation studies described above was followed.

For enhancer pretreatment, the same procedures described above for passive permeation were followed except that the buccal tissue was pretreated for 1 hour by adding 30 μl of chemical enhancer solution on top of buccal tissue in the donor compartment prior to the application of 0.3 ml ODAN.HCl gel.

Quantification of Ondansetron HCl

The concentration of ODAN.HCl in the receptor compartment was analyzed by HPLC. The system consisted of an Agilent HP 1100 series pump, a VWD detector and Agilent ChemStation for LC. A C18 column (150×4.6 mm C18 (2) 100 A Luna 5 μm, Phenomenex) with a guard column was used at 25° C. The mobile phase consisted of methanol and PBS (pH=7.5) at 65:35 (Zheng, 2002). The flow rate was 1.0 ml/minute and the drug was detected at 310 nm. The injection volume was 20 μl. The linear range was 5.36-107.2 μg/ml (r=0.9994). The detection limit was 0.107 μg/ml and daily RSD≦±3.0%.

Histology of Tissues

The morphological changes in both untreated and treated buccal tissues were evaluated using light microscopy. Buccal membrane samples were sectioned carefully and fixed in 10% buffered formalin for 1 day at room temperature. Tissue samples were successively dehydrated with 50%, 75, 95%, and 100% alcohol for one hour each. This was followed by immersing in xylene at least three times, and finally embedding in Tissue-Tek O.C.T. compound under dry ice. Using a microtome (Leica Model CM 1850, Leica Microsystems, Inc., Bannockburn, Ill.), 7 μm thin slices were prepared and then stained with Mayer's Harris Hematoxylin and Eosin Y (H&E). The stained slices were examined under a Nikon Eclipse E 800 light microscope (Micro Optics, Cedar Knolls, N.J.) at 40×. A Nikon Digital Camera (Model DXM 1200) was used to capture images. Images were processed by SPOT™ Imaging Software, Version 5.0 (Diagnostic Instrument, Inc., Sterling Heights, Mich.).

Buccal Tissue Cytotoxicity Study

MTS 3-(4,5-dimethylthiazol-2-yl)-5-(3-carboxymethoxyphenyl)-2-(4-sulfophenyl)-2H-tetrazolium, inner salt) assay was used to evaluate enhancer cytotoxicity in buccal tissues. MTS assay is based on the ability of a mitochondrial dehydrogenase enzyme derived from viable cells to cleave the tetrazolium rings and form purple color formazan crystals that are largely impermeable to cell membranes, thus resulting in their accumulation within healthy cells (Promega Corp., 2009). The number of surviving cells is directly proportional to the level of the formazan. The color can then be quantified at 490 nm using a Microplate Power Wave X Scanning Spectrophotometer (Bio-TEK Instruments, Inc., Winooski, Vt.).

EpiOral™ tissue (ORL-200) was used, which is a multilayered tissue mainly composed of an organized basal layer and multiple non-cornified layers analogous to native human buccal tissue. A 24-well plate containing ORL-200 (cell culture inserts) was stored in the refrigerator (4° C.) prior to use. Under sterile forceps, the cell culture inserts were transferred into four 6-well plates containing pre-warmed assay medium (37° C.). The 6-well plates containing the tissue samples were then placed in a humidified 37° C. and 5% CO₂ incubator for 1 hour prior to dosing. Tissues were exposed to 20, 60 and 240 min. of enhancer solution dosed in duplicate. Two inserts were left untreated to serve as a Negative Control (sterilized water) and another two inserts served as a Positive Control (1% Triton X-100—a nonionic surfactant, polyethylene glycol p-(1,1,3,3-tetramethylbutyl)-phenyl ether). Exposure time for the Positive and Negative Controls was 60 min. as per EpiOral 200 Protocol from MatTek Corp. (MatTek, 2009). After 1 hour incubation, the assay media was removed from the wells and replaced with 0.9 ml of pre-warmed fresh media, then 40 μl of 1:1 diluted enhancer solutions in sterilized water were added into the cell culture inserts atop the EpiOral™ tissue. 40 μl of sterilized water as negative control and 100 μl of 1% Triton-100 as positive control were added in separate wells. Then the well plates containing the dosed EpiOral™ tissues were returned to the incubator for 20, 60, and 240 min. After the exposures, each tissue insert was gently removed, rinsed with PBS solution at least twice and transferred into a 24-well plate containing premixed MTS solution (ratio of MTS reagent:assay medium=1:4). The 24-well plate was then returned to 37° C., 5% CO, incubator for 3 hours. After this, 100 μl of the reacted MTS solution from each well was pipetted into a marked 96-well microtiter plate for spectrophotometer reading (SPR) at 490 nm using Microplate Power Wave X Scanning Spectrophotometer (Bio-TEK Instruments, Inc., Winooski, Vt.). 100 μl of assay medium was used as a blank. The EpiOral tissue % viability at each of the dosed concentrations was calculated using the following formula:

% Viability=100×(SPR for Treated Sample/SPR for Negative Control).

Dose response curve was established using a semi-log scale to plot % viability (linear y axis) vs. the dosing time (log x axis). ET-50 value—the time required for the % viability of EpiOral™ tissue to fall to 50 was obtained through interpolation. All the SPR were deducted from blank readings for viability and ET-50 value final calculations.

### Results and Discussion

### The Effect of Current on Transbuccal Delivery of ODAN.HCl

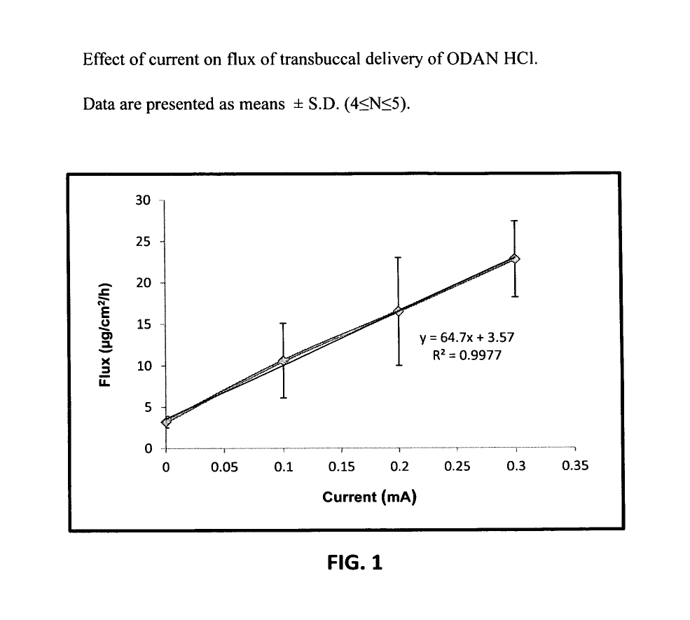

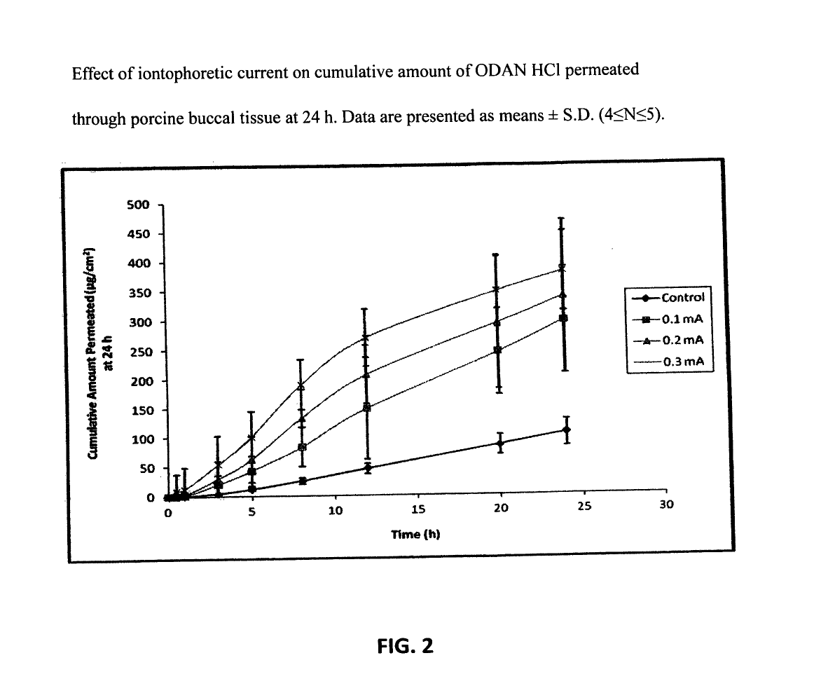

Anodal iontophoresis at 0.1, 0.2, and 0.3 mA was applied to buccal tissue for 8 hours (Stage I) and then discontinued to allow passive permeation of drug for another 16 hours (Stage II-8 to 24 hrs.). The effect of current on the transbuccal delivery of ODAN.HCl flux, cumulative amount of drug permeated and ER are shown in Tables 5 and 6 for Stage I (0 to 8 hrs.) and Stage II (8 to 24 hrs.). Iontophoresis (0.1, 0.2 and 0.3 mA) provided significantly higher flux of ODAN.HCl when compared to control (untreated) (p<0.05). The transbuccal flux linearly increased as current increased from 0.1 to 0.3 mA (FIG. 1). FIG. 2 shows the cumulative drug amount permeated from 0-24 hours. It indicates that the enhancement effect of iontophoresis was significant not only during the 8 hours of treatment but throughout the 24 hour of the study. Furthermore, the enhancement ration increased as current increased at Stage I. The enhancement ratio at Stage II leveled off but was still significantly higher than that of control.

TABLE 5
Effect of current on transbuccal delivery of ODAN•HCl at Stage I.

| Iontophoresis | Flux | Q₈ | |
|---|---|---|---|
| (mA) | (μg/cm²/h) | (μg · cm²) | ER |
| Control | 3.2 ± 0.7 | 25.5 ± 5.1 | 1 |
| 0.1 | 10.6 ± 4.5^{b} | 83.3 ± 33.5 | 3.3 |
| 0.2 | 16.5 ± 6.5^{b} | 132.7 ± 50.1 | 5.2 |
| 0.3 | 22.8 ± 4.6^{b} | 190.4 ± 42.7 | 7.1 |

^{a}Data are presented as means ± S.D. (4 ≦ N ≦ 5).
^{b}Statistically significantly higher than control at p < 0.05 (Student's t-test).

TABLE 6
Effect of Current on Transbuccal
delivery of ODAN•HCl at Stage II

| Iontophoresis | Flux | Q₂₄ | |
|---|---|---|---|
| (mA) | (μg/cm²/h) | (μg · cm²) | ER |
| Control | 4.9 ± 1.1 | 104.7 ± 22.8 | 1 |
| 0.1 | 13.7 ± 4.3^{b} | 296.9 ± 90.1 | 2.8 |
| 0.2 | 12.7 ± 5.3^{b} | 337.4 ± 130.5 | 2.6 |
| 0.3 | 11.9 ± 2.3^{b} | 380.4 ± 68.1 | 2.4 |

^{a}Data are presented as means ± S.D. (4 ≦ N ≦ 5).
^{b}Statistically significantly higher than control at p < 0.05 (Student's t-test).

### Effect of Chemical Enhancers on Transbuccal Delivery of ODAN.HCl

Azone in propylene glycol (PG), DDAIP.HCl in water, DDAIP.HCl in PG, DDAIP in PG, Br-iminosulfurane in PG or the vehicle PG alone was applied (30 μl) to the buccal tissue for 1 hour prior to the permeation experiment. After the 1 hour enhancer pretreatment, 0.3 ml of 0.5% ODAN.HCl gel formulation was applied. Samples were taken at different time points from 0 to 24 hours.

Tables 7 and 8 compare flux and ER of passive transport of ODAN.HCl through enhancer pretreated and untreated (control) tissues. The passive flux of ODAN.HCl was significantly greater in all enhancer treated tissues in comparison to control (p<0.05). DDAIP.HCl in water resulted in significantly higher flux and ER than did DDAIP in PG, Azone in PG and Br-iminosulfurane in PG (p<0.05).

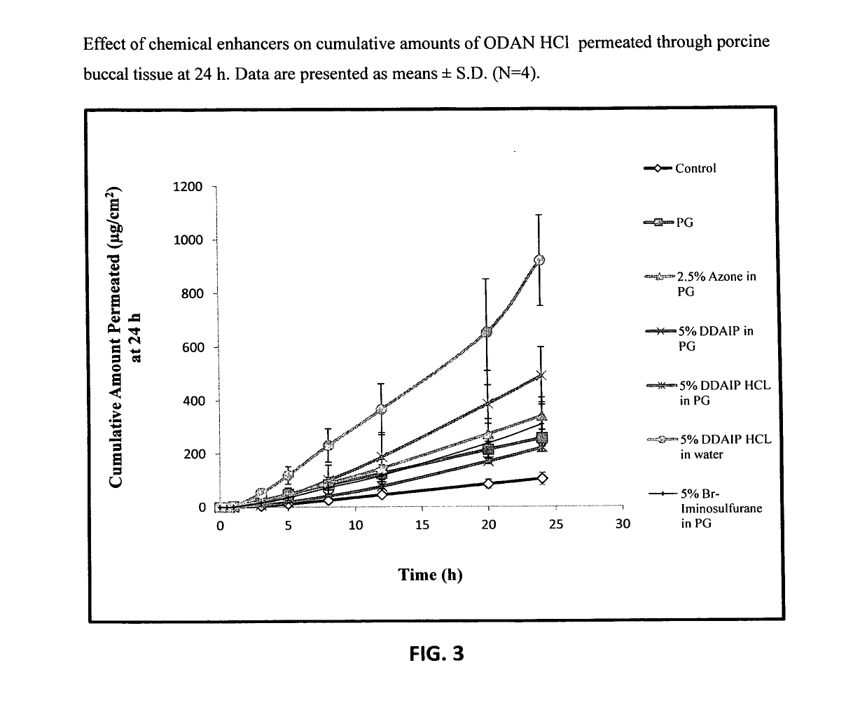

FIG. 3 shows the cumulative amount of ODAN.HCl permeated through tissue from 0-24 hours. It shows that compared to the control, the enhancement effect of chemical enhancers was significant throughout the 24 hour of the study. DDAIP.HCl in water exhibited significantly higher permeability than DDAIP.HCl in PG (p<0.05), indicating that PG actually acted as a penetration “retardant” when used as a vehicle for DDAIP.HCl. The enhancement differences among the four emhancers may be due to their different properties and mechanisms of action.

Azone is a hydrophobic enhancer which is reported to increase lipid fluidity and enhances only intercellular drug diffusion. Hydrophobic enhancer Br-iminosulfurane is believed to be more effective in enhancing hydrophobic drug permeation through lipid membranes. DDAIP reportedly enhances drug transport by interacting with the polar region of the phospholipid bilayer and promoting the motional freedom of lipid hydrocarbon. However, buccal tissue is non-keratinized, lacks the organized intercellular lipid lamellae and contains large mounts of polar lipids that allow more interaction with hydrophilic compounds. The hydrophilic DDAIP.HCl was more potent in enhancing transbuccal delivery of a hydrophilic drug through both intercellular (paracellular) and intracellular (transcellular) pathways than hydrophobic enhancers Azone, DDAIP and Br-iminosulfurane. Furthermore, DDAIP.HCl pretreatment alone provided significantly higher enhancement of transbuccal delivery of ODAN.HCl than iontophoresis at 0.3 mA during the first 8 hours and the following 16 hours of study (p<0.05).

Table 7 shows that using 5% DDAIP.HCl in water treatment, transbucal delivery of ODAN.HCl (Q₂₄) could reach 920.3 (μg/cm²) within 24 h, i.e. potentially when a small patch of 10 cm² containing only 0.5% ODAN.HCl is used, this particular enhanced drug delivery system could deliver 9.2 mg/day into blood circulation through buccal route.

TABLE 7
Effect of Chemical Enhancers on Transbuccal
Delivery of ODAN•HCl at Stage I^{a}

| | Flux | Q₈ | |
|---|---|---|---|
| Chemical Enhancers | (μg/cm²/h) | (μg · cm²) | ER |
| Control | 3.2 ± 0.7 | 25.5 ± 5.1 | 1 |
| Propylene Glycol (PG) | 10.7 ± 2.6^{b} | 83.4 ± 19.3 | 3.3 |
| 2.5% Azone in PG | 11.3 ± 2.9^{b} | 88.7 ± 23.1 | 3.5 |
| 5.0% DDAIP in PG | 5.1 ± 1.1 | 41.5 ± 8.1 | 1.6 |
| 5.0% DDAIP•HCl in water | 29.3 ± 8.0^{c} | 231.2 ± 62.7 | 9.2 |
| 5.0% DDAIP•HCl in PG | 12.4 ± 7.0^{b} | 100.7 ± 56.4 | 3.9 |
| 5.0% Br-Iminosulfurane in PG | 9.2 ± 3.6^{b} | 73.1 ± 27.8 | 2.9 |

^{a}Data are presented as means ± S.D. (N = 4).
^{b}Statistically significantly higher than control at p < 0.05 (Student's t-test).
^{c}Statistically significantly higher than the other enhancer treated and control at p < 0.05 (ANOVA)

TABLE 8
Effect of chemical enhancers on transbuccal
delivery of ODAN•HCl at Stage II^{a}

| | Flux | Q₂₄ | |
|---|---|---|---|
| Chemical Enhancers | (μg/cm²/h) | (μg · cm²) | ER |
| Control | 4.9 ± 1.1 | 104.7 ± 22.8 | 1 |
| PG | 10.9 ± 0.8^{b} | 257.1 ± 31.9 | 2.2 |
| 2.5% Azone in PG | 15.8 ± 3.1^{b} | 340.7 ± 70.0 | 3.2 |
| 5.0% DDAIP in PG | 11.3 ± 0.8^{b} | 221.0 ± 15.6 | 2.3 |
| 5.0% DDAIP•HCl in water | 41.6 ± 7.6^{c} | 920.3 ± 169.1 | 8.5 |
| 5.0% DDAIP•HCl in PG | 24.5 ± 3.8^{b} | 490.8 ± 107.2 | 5.0 |
| 5.0% Br-Iminosulfurane in PG | 14.8 ± 4.1^{b} | 309.5 ± 83.1 | 3.0 |

^{a}Data are presented as means ± S.D. (N = 4).
^{b}Statistically significantly higher than control at p < 0.05 (Student's t-test).
^{c}Statistically significantly higher than the other enhancer treated and control at p < 0.05 (ANOVA)

### Effect of Combined Treatment of Chemical Enhancers and Iontophoresis on Transbuccal Delivery of ODAN.HCl

Azone in PG, DDAIP.HCl in water, DDAIP.HCl in PG, DDAIP in PG, Br-iminosulfurance in PG and vehicle PG was applied (30 μl) to the top of buccal tissue for 1 h prior to the anodal iontophoretic permeation experiment. After 1 hour enhancer pretreatment, 0.3 ml of 0.5% ODAN.HCl gel formulation was applied to the top of buccal tissues, and then 0.3 mA iontophoresis was applied for 8 h. At the end of 8 hours of 0.3 mA iontophoresis treatment, iontophoresis was ceased to allow passive permeation to continue for another 16 hours. Samples were taken at different time points from 0 to 24 hours.

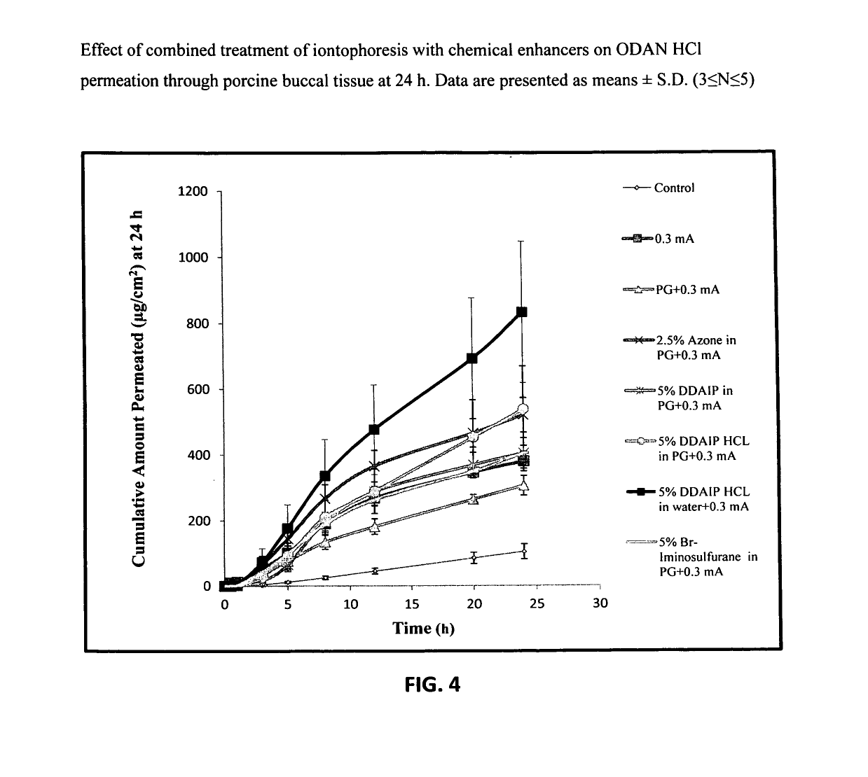

Tables 9 and 10 show that combined treatment of enhancer with iontophoresis provided significantly higher permeability than that of control (p<0.05) and the combination of DDAIP.HCl in water and iontophoresis (0.3 mA) was the most effective treatment in enhancing transbuccal delivery of ODAN.HCl (FIG. 4). However, with DDAIP.HCl in water pretreatment, the flux (30.2 μg/cm²/h) from the combined treatment was much less than the sum of the fluxes of DDAIP.HCl in water (41.6 μg/cm²/h) and iontophoresis (11.9 μg/cm²/h) during the 24 h o the study. The same trend was recorded for DDAIP.HCl in PG. DDAIP.HCl—the salt form of DDAIP contained ions that appears to compete with ODAN.HCl for iontophoresis, thus reducing the enhancement effect of iontophoresis.

TABLE 9
Effect of Combined Treatment of Current and Chemical Enhancers
on Transbuccal delivery of ODAN•HCl at Stage I^{a}

| | Flux | Q₈ | |
|---|---|---|---|
| Chemical Enhancers | (μg/cm²/h) | (μg · cm²) | ER |
| Control | 3.2 ± 0.7 | 25.5 ± 5.1 | 1 |
| 0.3 mA | 22.8 ± 4.6^{b} | 190.4 ± 42.7 | 7.1 |
| PG + 0.3 mA | 19.7 ± 1.2^{b} | 133.9 ± 10.8 | 4.6 |
| 2.5% Azone in PG + 0.3 mA | 34.1 ± 6.0^{b} | 267.9 ± 42.2 | 10.7 |
| 5.0% DDAIP in PG + 0.3 mA | 23.5 ± 1.6^{b} | 196.3 ± 9.1 | 7.3 |
| 5.0% DDAIP•HCl + 0.3 mA | 43.0 ± 14.6^{b} | 336.7 ± 110.7 | 13.4 |
| In water | | | |
| 5.0% DDAIP•HCl in PG + | 26.1 ± 4.2^{b} | 210.8 ± 52.8 | 8.2 |
| 0.3 mA | | | |
| 5.0% Br-Iminosulfurane | 24.0 ± 3.6^{b} | 188.6 ± 25.1 | 7.5 |
| in PG + 0.3 mA | | | |

^{a}Data are presented as means ± S.D. (3 ≦ N ≦ 5).
^{b}Statistically significantly higher than control at p < 0.05 (Student's t-test).

TABLE 10
Effect of Combined Treatment of Current and Chemical Enhancers
on Transbuccal delivery of ODAN•HCl at Stage II^{a}

| | Flux | Q₂₄ | |
|---|---|---|---|
| Treatment | (μg/cm²/h) | (μg · cm²) | ER |
| Control | 4.9 ± 1.1 | 104.7 ± 22.8 | 1 |
| 0.3 mA | 11.9 ± 2.3^{b} | 380.4 ± 68.1 | 2.4 |
| PG +0.3 mA | 10.7 ± 1.5^{b} | 306.9 ± 15.0 | 2.0 |
| 2.5% Azone in PG + 0.3 mA | 15.1 ± 0.5^{b} | 520.9 ± 52.7 | 3.1 |
| 5.0% DDAIP in PG + 0.3 mA | 12.5 ± 3.1^{b} | 405.0 ± 46.2 | 2.6 |
| 5.0% DDAIP•HCl + 0.3 mA | 30.2 ± 7.7^{b} | 833.5 ± 214.4 | 6.2 |
| in water | | | |
| 5.0% DDAIP•HCl in PG + | 20.5 ± 5.2^{b} | 538.8 ± 131.4 | 4.2 |
| 0.3 mA | | | |
| 5.0% Br-Iminosulfurane | 13.0 ± 2.5^{b} | 405.3 ± 22.7 | 2.7 |
| in PG + 0.3 mA | | | |

^{a}Data are presented as means ± S.D. (3 ≦ N ≦ 5).
^{b}Statistically significantly higher than control at p < 0.05 (Student's t-test).

### Histological Study

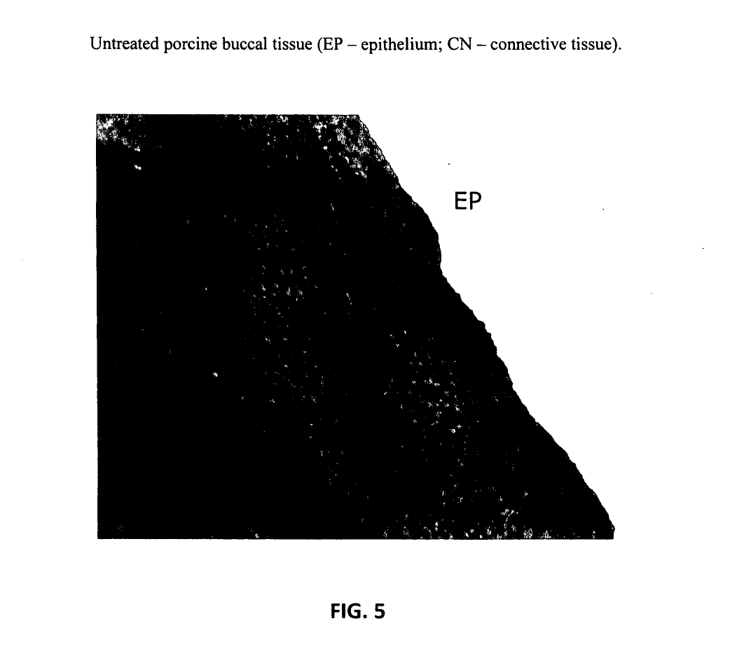

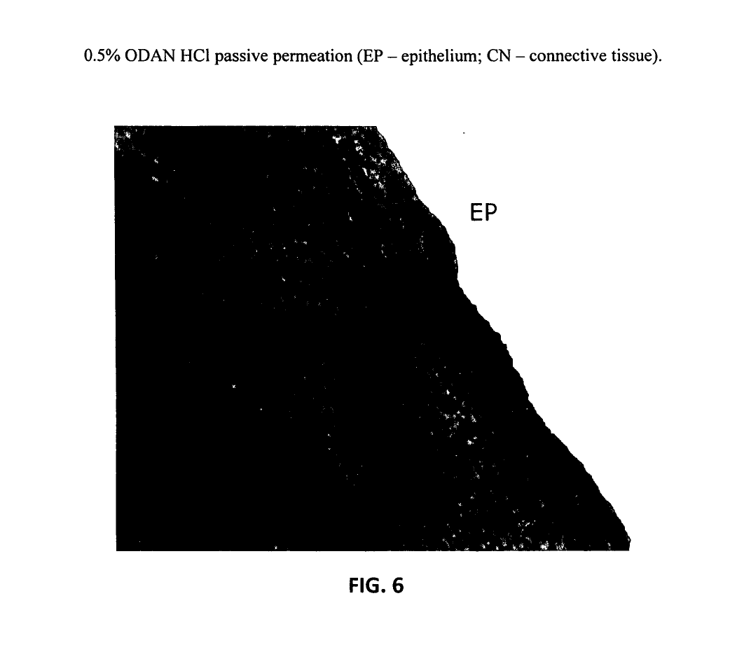

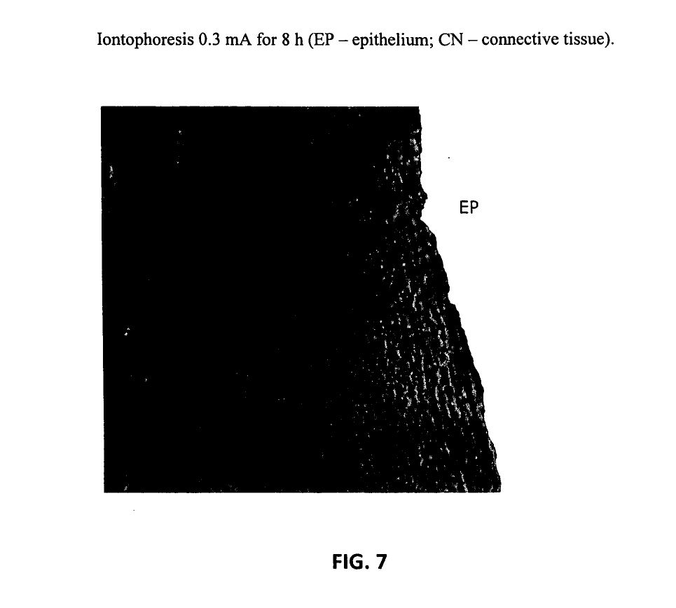

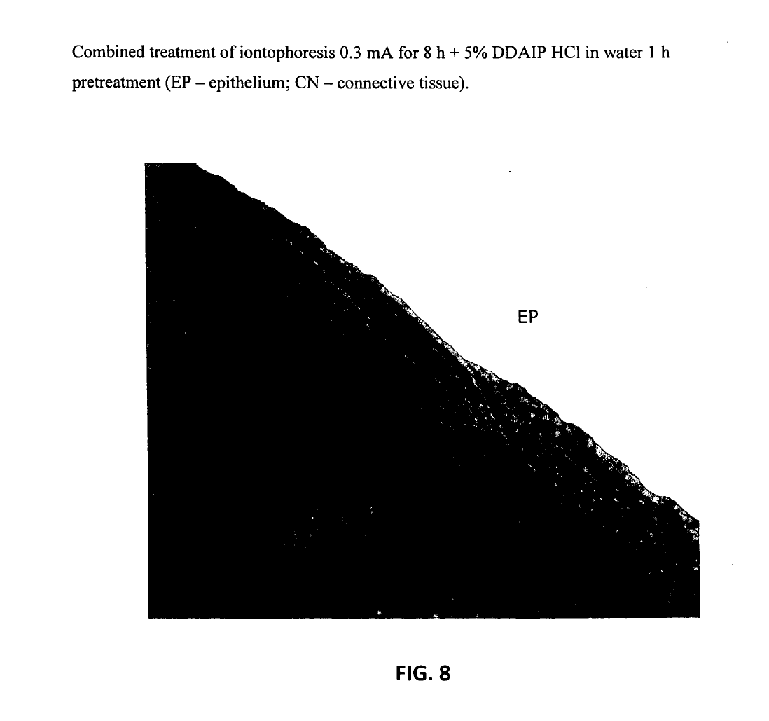

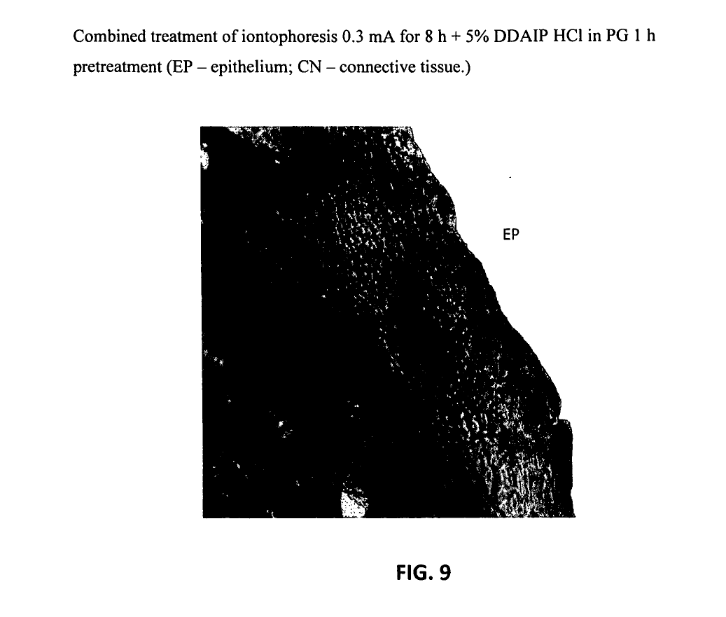

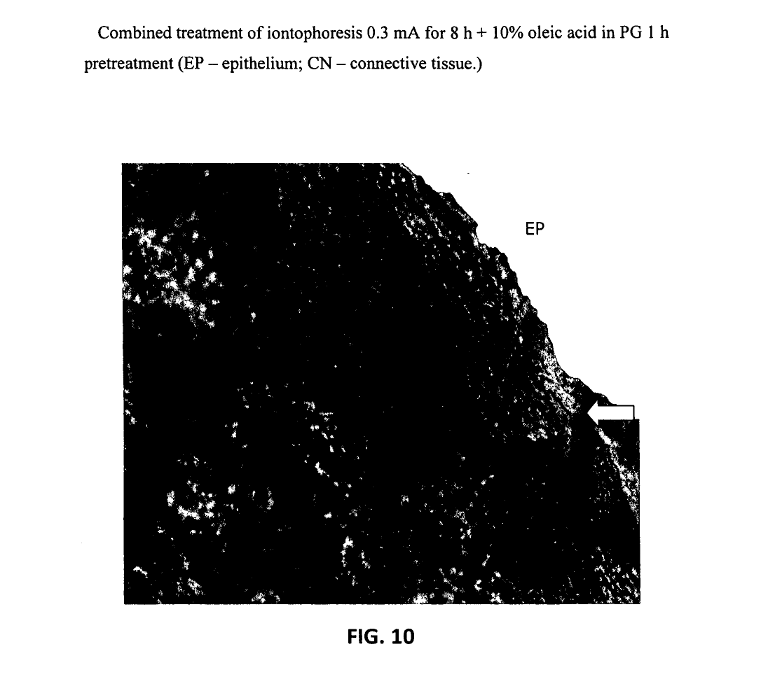

A histological study was performed to evaluate the integrity of treated and untreated porcine tissues using standard H&E methodology. Treated tissues included those following 0.3 mA iontophoresis for 8 h and combined treatment of 0.3 mA iontophoresis for 8 h, plus 1 h enhancer pretreatment: DDAIP.HCl in water and DDAIP.HCl in PG. Light micrographs (40×) (FIG. 5-10) show the morphology of treated and untreated buccal tissues. Compared to untreated (FIG. 5), no major morphological changes were observed after 0.5% ODAN.HCl passive permeation (FIG. 6), 0.3 mA for 8 h (FIG. 7), 0.3 mA for 8 h+5% DDAIP.HCl in water treatment (FIG. 8), and 0.3 mA for 8 h+5% DDAIP.HCl in PG treatment (FIG. 9). 10% Oleic acid in PG pretreatment was used as a positive control since it was preported to cause detachment of keratinocytes in stratum corneum of skin. Thus a similar approach was taken and 10% Oleic acid in PG pretreatment was used as a positive control and integrity of the treated tissue was recorded. The micrograph showed significant damage in the buccal epithelial layers—the white arrow pointed area (FIG. 10).

### EpiOral™ Cytotoxicity Study

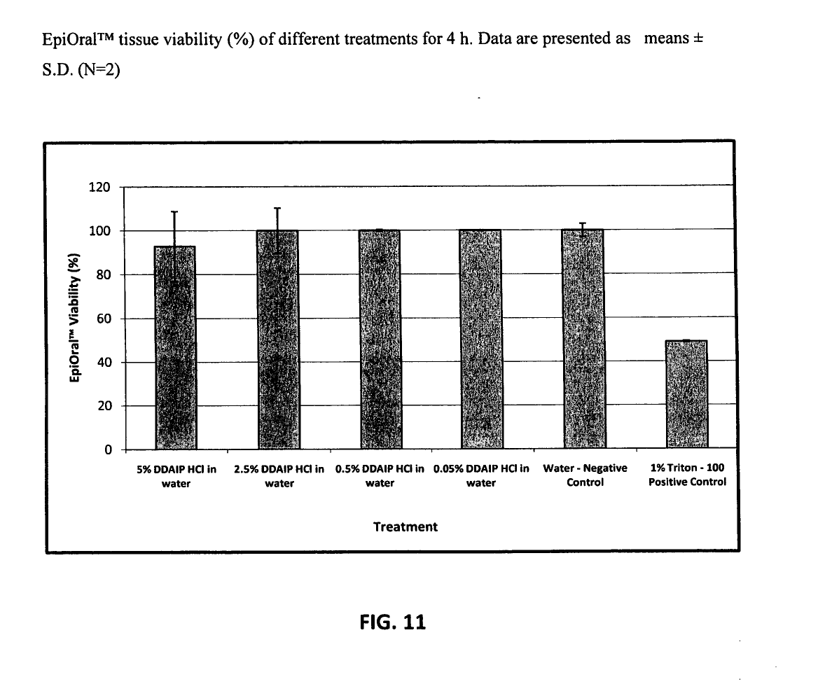

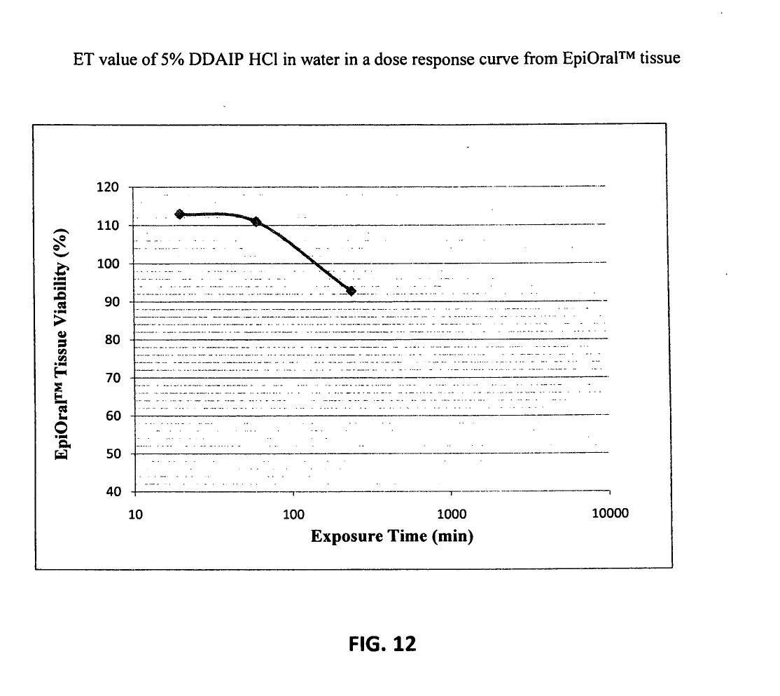

Cytotoxicity evaluation (MTS assay) was conducted using EpiOral™ tissue in duplicate using 5% DDAIP.HCl in water—the best performing chemical enhancer from this study. Sterilized water treated issue was used as negative control and 1% Triton—100 treated tissue as positive control. At the end of the experiments, cell viability was evaluated by measuring the mitochondrial dehydrogenase activities according to the MTS assay (Promega Corp., 2009). The mean optical density (OD) of the untreated control tissues was set to represent 100% of viability (MTS test, N=2, OD=0.999) and the results were qualified as percentage of the negative controls. FIG. 11 demonstrates that DDAIP.HCl treatment in a concentration range of 0.05% to 5% in water for 4 h did not reduce the viability of EpiOral™ tissue compared to water—the negative control, and viability (100%) of 5% DDAIP.HCl in water treated EpiOral tissue was significantly higher than that (49%) of positive control. The DDAIP.HCl in water dose response curve obtained from MTS EpiOral™ tissue (FIG. 12) indicated that ET-50 value of 5% DDAIP.HCl in water was greater than 1000 min, significantly more than the 49 min for the positive control, indicating that at concentrations up to 5% in water, DDAIP.HCl is potentially safe to use for transbuccal drug delivery.

Both iontophoresis (0.1, 0.2, 0.3 mA) or DDAIP.HCl pretreatment can provide significantly higher permeability for ODAN.HCl across porcine buccal tissues compared to control (p<0.05) while Azone, DDAIP and Br-iminosulfurane were only marginally effective. The 5% DDAIP.HCl in water produced no major morphological changes in porcine buccal tissue and was the most effective enhancer/vehicle formulation for transbuccal delivery of ODAN.HCl.

### Example 7

The effects of iontophoresis, chemical enhancers and their combined treatments on transdermal and transbuccal delivery of LHCl, NHT and DHCl were evaluated. The chemical enhancers used were DDAIP and DDAIP.HCl, and Br-iminosulfurane. DDAIP, DDAIP.HCl and Br-iminosulfurane at <5% are considered to be low toxic and biodegradable. A popular enhancer −1 dodecylazacycloheptan-2-one (Azone, laurocapram) was used as a control. No comparison was made between transdermal and transbuccal drug delivery using iontophoresis or the combined treatment of chemical enhancers and iontophoresis.

Lidocaine HCl (LHCl), Nicotine Hydrogen Tartrate (NHT) and Dilitiazem HCl (DHCl) gel compositions were prepared as described below.

Cellulose gum was dispersed in water first, then the selected amount of drug (2%) was added and mixed well using lightning mixer until uniform to obtain separate LHCl, NHT and DHCl gel formulations, respectively as shown in Table 11.

TABLE 11
Lidocaine HCl, nicotine hydrogen tartrate
and dilitazem HCl gel formulations

| | Formulations (w/w %) | | |
|---|---|---|---|
| | 2.5% | 2% Nicotine | 2% |
| | Lidocaine | Hydrogen | Diltiazem |
| Ingredients | HCl Gel | Tartrate Gel | HCl Gel |
| Lidocaine HCl | 2.5 | | |
| Nicotine Hydrogen | | 2.0 | |
| Tartrate | | | |
| Diltiazem HCl | | | 2.0 |
| Cellulose Gum | 2.0 | 2.0 | 1.0 |
| Water | 95.5 | 96.0 | 97.0 |
| pH | 6.0 | 4.0 | 6.0 |
| Viscosity (cps) | 9000 | 9200 | 800 |

Skin and Buccal Tissue Preparation

Porcine skin with a thickness of about 500 to 600 μm obtained from young Yorkshire pigs (3-4 months old; 25-30 Kg) was prepared using Padgett® Model B Electric Dermatome (Integra LifeSciences, Plainsboro, N.J.). The dermatomed skin was then cut into a size of 1.0 cm² and stored at −80° C. no more than 3 months prior to use. In the beginning of a permeation study, at room temperature the skin was defrosted first and then soaked in Phosphate Buffer Saline (PBS) solution for one hour.

Buccal mucosa samples were harvested from pig's cheek area and placed below −30° C. The tissue samples were defrosted at room temperature first before use. Then a scalpel blade and a surgical scissor were used to remove the underlying connective tissue and trim the buccal mucosa to about 300 to 400 μm in thickness. Before each evaluation the buccal tissues were submerged in PBS (pH 7.5) for 1 hour.

Preparation of Anodal and Cathodal Electrodes

Anodal electrodes (Ag) were prepared using pure silver (Ag) wire (0.r mm in diameter). Cathodal electrodes (AgCl) were made by connecting AgCl powder coating Ag wires and pure Ag wires partially dipped in 0.1 N HCl solution to a power source of 3 mA for 12 hours.

Enhancer Solution Preparation

5% w/v DDAIP, 5% Br-imminosulfurane and 2% w/v Azone enhancer were prepared using PG as the vehicle 5% w/w DDAIP.HCl in PG and water solutions were prepared using water and PG as separate vehicles

In Vitro Transdermal and Transbuccal Permeation Study

In vitro transdermal and transbuccal drug permeation studies were conducted using Franz diffusion cells porcine skin and buccal tissues. The following studies were performed: passive (control) permeation with 1 hour enhancer pretreatment, permeation with 8 hour iontophoresis (0.1 or 0.3 mA) treatment, and permeation with 1 hour enhancer pretreatment plus 8 hour iontophoresis (0.3 mA) treatment. At 37° C., the duration for all studies was 8 hours.

For the passive in vitro permeation study, PBS (pH 7.5) solution was added into Franz cell receptor compartment and stirred at 600 rpm. The skin or buccal tissue was sandwiched between donor and receptor compartments with the side of epidermal or connective tissue attached to the receptor compartment. The available diffusion area was 0.64 cm². 0.3 ml of each tested gel formulation was added into the donor compartment at the start of each experiment. At each time points (0.0, 0.5, 1, 3, 5, or 8 hours), 300 μl sample were taken from the receptor compartment for HPLC sample analysis and then quickly filled with an exact amount of 300 μl PBS (pH 7.5) (Diaz del Consuelo, et al., 2005; Jacobsen, 2001; Kulkarmi, et al., 2010; Send and Hincal, 2001).

For permeation study with enhancer pretreatment, the skin or buccal tissue was treated first for 1 hour by added 30 μl of chemical enhancer solution on top of skin or buccal tissue in the donor compartment before the addition of a tested gel formulation. Then the same procedures described above for passive permeation studies were followed.

For iontophoresis studies, 0.1 and 0.3 mA for 8 hours of treatment was provided by Phoresor II Auto (Model PM 850). The anodal electrode (Ag) was submerged in the gel formulation in the donor compartment, but stayed about 2 mm above the skin or buccal tissue. The cathode electrode (AgCl) was placed into the receptor compartment. The anodal and cathode electrodes were connected to the positive and negative terminators of Phoresor II Auto power source to conduct iontophoresis treatment on skin or buccal tissue. Iontophoresis was terminated after 8 hour application. The same sampling method and time points were used as described above for passive permeation experiments.

HPLC Analysis of LHCl, NHT and DHCl

An Agilent HP 1100 HPLC system with a VWD detector and Agilent ChemStation for LC were used to analyze LHCl, NHT, and DHCl concentrations (as shown in Table 12) in the receptor compartment at different time points.

TABLE 12
HPLC Methods for Analysis of Lidocaine HCl, nicotine hydrogen tartrate and dilitizem HCl

| Drug | HPLC Column | HPLC Conditions | Mobile Phase |
|---|---|---|---|
| Lidocaine HCl | Waters column | Flow rate: 1.5 mL/min. | 35 ml glacial acetic acid (99%) |
| | Nova-Pak C18 column | Column temp.: 25° C. | 930 ml deionized water; |
| | 4 μm 3.9 × 300 min. | UV wavelength: 254 nm | adjusted pH = 3.4 using 1N |
| | | Injection volume: 15 μl | NaOH solution; 4 volume of |
| | | | the above solution plus 1 volume |
| | | | of acetonitrile |
| Nicotine | Phenomenex column | Flow rate: 1.4 ml/min. | 5 Phosphate Buffer saline |
| Hydrogen | 150 × 46 mm C18 (2) | Column temp.: 25° C. | (PBS) tablets; 1000 ml |
| Tartrate | 100 A Luna 5 μm | UV wavelength: 256 nm | water; 7.5 mL triethylamine |
| | | | adjusted pH = 6.8 using glacial |
| | | | acetic acid (99%); 500 mL |
| | | | methanol |
| Diltiazem HCl | Phenomenex column | Flow rate: 1.0 ml/min. | Glacial acetic acid aqueous |
| | 150 × 46 mm C18 (2) | Column temp.: 25° C. | solution (pH = 3.0):methanol = 1:4; |
| | 100 A Luna 5 μm | UV wavelength: 310 nm | triethylamine to adjust pH |
| | Phenyl-hexyl | Injection volume: 20 μl | to 6.8 |

Data Analyses

Steady state flux at time t (J. μg cm⁻²) was represented by the slope of the linear section of the plot of cumulative drug amount permeated vs. time. Q₈ (p,g cm⁻²) was defined as the cumulative drug amount permeated into the receptor compartment at 8 hour from the drug formulation in the donor compartment. The enhancement ratio (ER) for flux was obtained from the following formula:

$$\frac{\begin{matrix}ER = Flux for treated skin or buccal tissue with enhancer \\ or iontophoresis or their combination\end{matrix}}{flux for treated and untreated skin or buccal tissue}$$

Results were demonstrated as mean±standard deviation (S.D.) (n) where n was the number of experiment replicates. The unpaired Student's t-test was used to analyze the difference between fluxes for treated tissue and untreated (control) tissue. ANOVA was used to compare fluxes among different treated tissues, and a difference with p<0.05 was considered to be statistically significant.

### Results and Discussion

Effect of Iontophoretic Treatment on Transdermal and Tranbuccal Delivery of LHCl, NHT and DHCl.

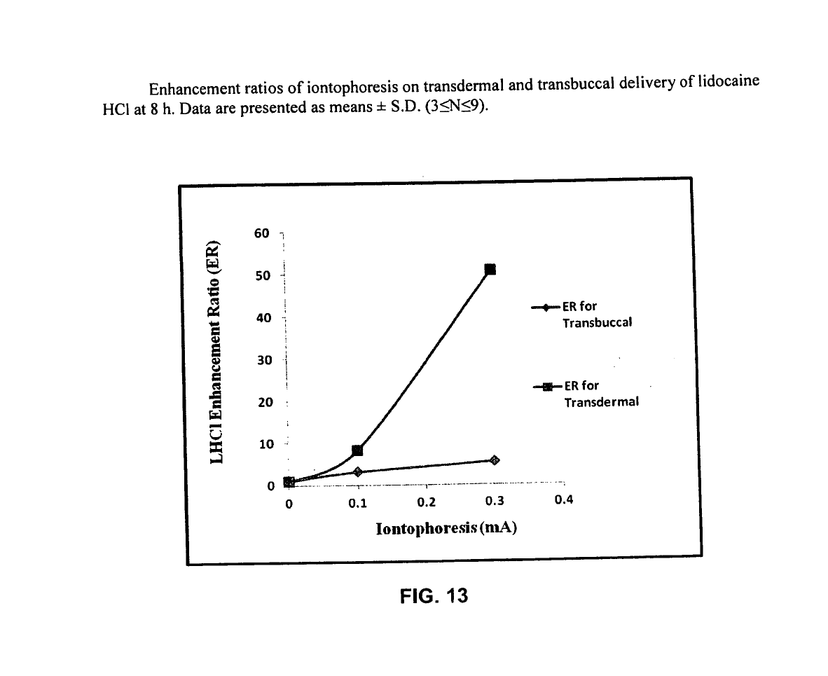

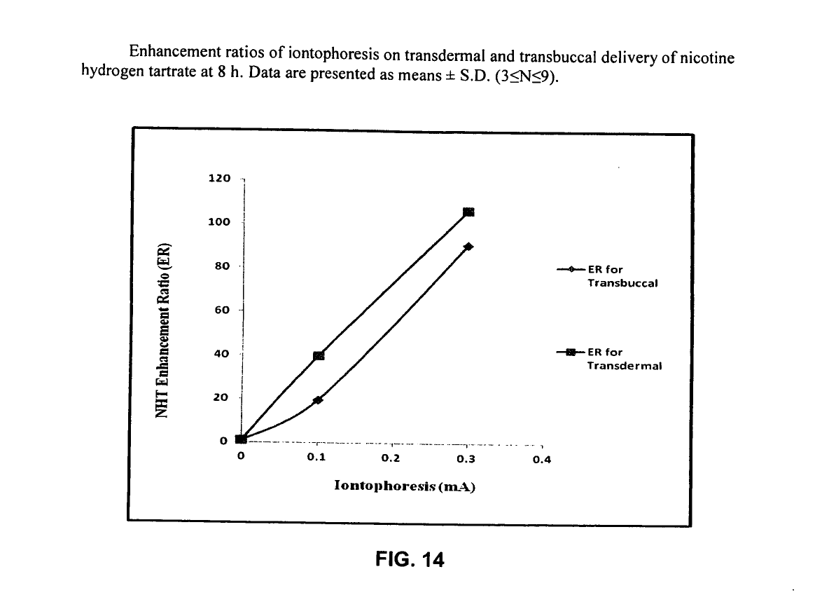

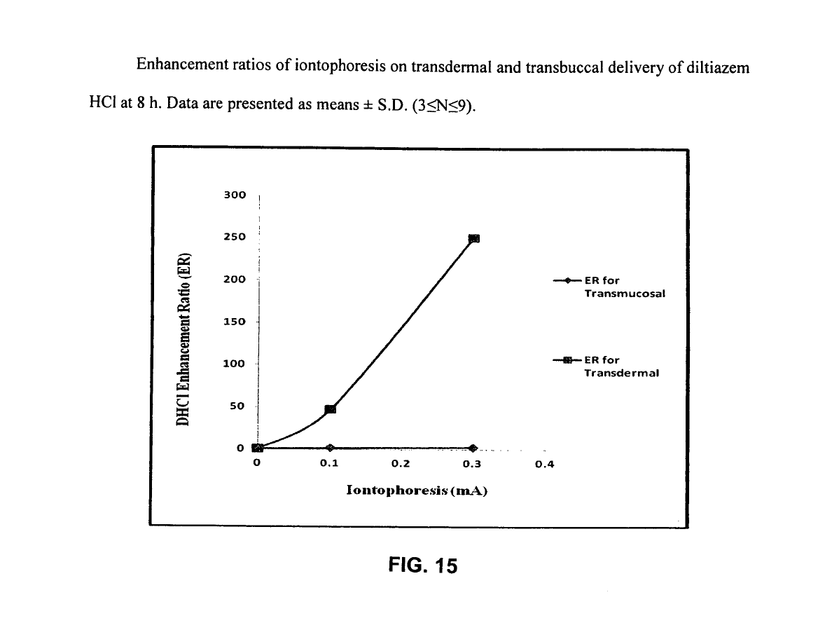

Anodal iontophoresis (0.1 mA or 0.3 mA) treatment was conducted on porcine skin and buccal tissue for 8 hours. Tables 13-15 and FIGS. 13-15 show the results of the flux, cumulative amount of drug permeated and ER.

TABLE 13
Effect of 2 hr. iontophoresis treatment on transdermal
and transbuccal delivery of lidocaine HCl^{a}

| | Transdermal | | Transbuccal | |
|---|---|---|---|---|
| Treatment | Flux | Q₈ | Flux | Q₈ |
| (mA) | (μg/cm²*h) | (μg/cm²) | (μg/cm²*h) | (μg · cm²) |
| Control | 7.4 ± 5.8 | 59.7 ± 43.4 | 44.7 ± 9.6 | 345.6 ± 74.3 |
| 0.1 | 61.7 ± 20.8^{b} | 494.0 ± 152.0^{b} | 137.1 ± 13.1^{b} | 1085.2 ± 92.1^{b} |
| 0.3 | 375.6 ± 69.44^{c} | 2879.2 ± 531.1^{c} | 241.7 ± 60.5^{c} | 1910.2 ± 454.7^{c} |

^{a}Data are presented as means ± S.D. (3 ≦ N ≦ 9)
^{b}Statistically significantly higher than control (p < 0.05).
^{c}Statistically significantly higher than 0.1 mA and the control (p < 0.05)
Control - untreated passive; 0.1 mA ≈ 0.16 mA/cm²; 0.3 mA ≈ 0.47 mA/cm²
Q₈ - drug cumulative amount permeated within 8 hr.

TABLE 14
Effect of 8 hr. iontophoresis treatment on transdermal
and transbuccal delivery of nicotine hydrogen tartrate^{a}

| | Transdermal | | Transbuccal | |
|---|---|---|---|---|
| Treatment | Flux | Q₈ | Flux | Q₈ |
| (mA) | (μg/cm²*h) | (μg/cm²) | (μg/cm²*h) | (μg · cm²) |
| Control | 1.3 ± 1.9 | 9.9 ± 14.6 | 0.9 ± 0.4 | 6.9 ± 2.6 |
| 0.1 | 56.1 ± 11.4^{b} | 433.2 ± 85.4^{b} | 17.6 ± 6.9^{b} | 141.5 ± 58.6^{b} |
| 0.3 | 138.4 ± 72.3^{c} | 1326.6 ± 186.2^{c} | 81.7 ± 35.9^{c} | 629.5 ± 276.8^{c} |

^{a}Data are presented as means ± S.D. (3 ≦ N ≦ 9)
^{b}Statistically significantly higher than control (p < 0.05).
^{c}Statistically significantly higher than 0.1 mA and the control (p < 0.05)
Control - untreated passive; 0.1 mA ≈ 0.16 mA/cm²; 0.3 mA ≈ 0.47 mA/cm²
Q₈ - drug cumulative amount permeated within 8 hr.

TABLE 15
Effect of 8 hr. iontophoresis treatment on
transdermal and delivery of diltiazem HCl^{a}

| | Transdermal | | Transbuccal | |
|---|---|---|---|---|
| Treatment | Flux | Q₈ | Flux | Q₈ |
| (mA) | (μg/cm²*h) | (μg/cm²) | (μg/cm²*h) | (μg · cm²) |
| Control | 0.4 ± 0.3 | 3.0 ± 2.6 | 32.6 ± 9.5 | 258.3 ± 73.6 |
| 0.1 | 18.9 ± 10.4^{b} | 154.1 ± 83.5^{b} | 54.5 ± 2.6 | 430.0 ± 18.7^{b} |
| 0.3 | 100.3 ± 33.7^{c} | 796.8 ± 276.6^{c} | 80.7 ± 18.0^{b} | 650.9 ± 139.1^{b} |

^{a}Data are presented as means ± S.D. (3 ≦ N ≦ 9)
^{b}Statistically significantly higher than control (p < 0.05).
^{c}Statistically significantly higher than 0.1 mA and the control (p < 0.05)
Control - untreated passive; 0.1 mA ≈ 0.16 mA/cm²; 0.3 mA ≈ 0.47 mA/cm²
Q₈ - drug cumulative amount permeated within 8 hr.

The effect of iontophoresis (0.1 and 0.3 A) on the transdermal and transbuccal delivery of LHCl, NHT and DHCl was compared. During the same 8 hour period of permeation study, LHCl and DHCl passively diffused through porcine buccal tissue much more effectively than through porcine skin which was in agreement with published literature. But, it was noted that the difference between passive diffusion of transdermal and transbuccal delivery of NHT was not significant. When compared to the control, iontophoresis at 0.1 mA and 0.3 mA significantly enhanced both transdermal and transbuccal delivery of LHCl, NHT and DHCl.

It was noted that enhancement ratio (ER) from iontophoresis treatment (0.1 and 0.3 mA) on buccal tissue was consistently less than on skin tissue for the three tested drugs. This may be due to the fact that the major barrier of skin—SC has pores in hair shaft and eccrine gland areas that exhibit less resistance to ionized molecules. Meanwhile, compared to SC of skin, the major barrier of bucccal tissue—epithelium—contains no pores, small amounts of neutral of neutral lipids, but about 10 times more water and 8 times more polar lipids, mainly cholesterol sulfate and glucosylceramides, which may compete for iontophoresis, thus reduce the effect of iontophoresis on transbuccal drug delivery. As a result, when iontophoresis is applied, ionized compounds such as LHCL, NHT and DHCl may be transferred through hair shafts and eccrine glands more easily of skin than epithelium of buccal tissue, i.e., the impact of iontophoresis on transdermal delivery of LHCl, NET and DHCl was judged more significant than on transmucosal delivery.

Furthermore, for LHCl and DHCl, at 0.1 mA, flux and accumulative amount permeated at 8 hours for transbuccal delivery were higher than that of transdermal drug delivery. But at 0.3 mA, flux and accumulative amount permeated at 8 hours for transdermal delivery were higher than that of transbuccal drug delivery.

### Effect of Chemical Enhancers on Transdermal and Transbuccal Delivery of LHCl, NHT and DHCl

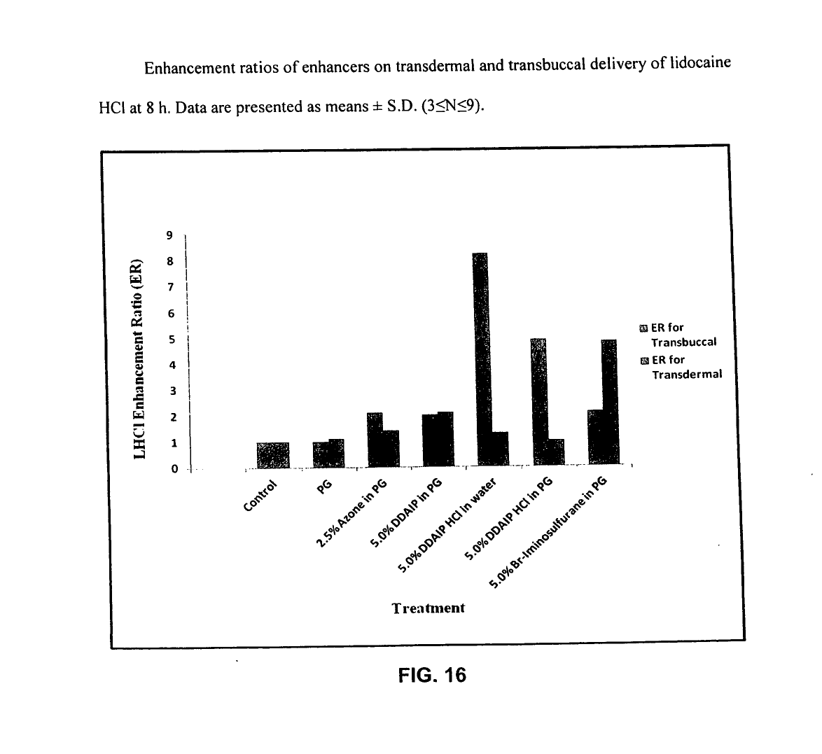

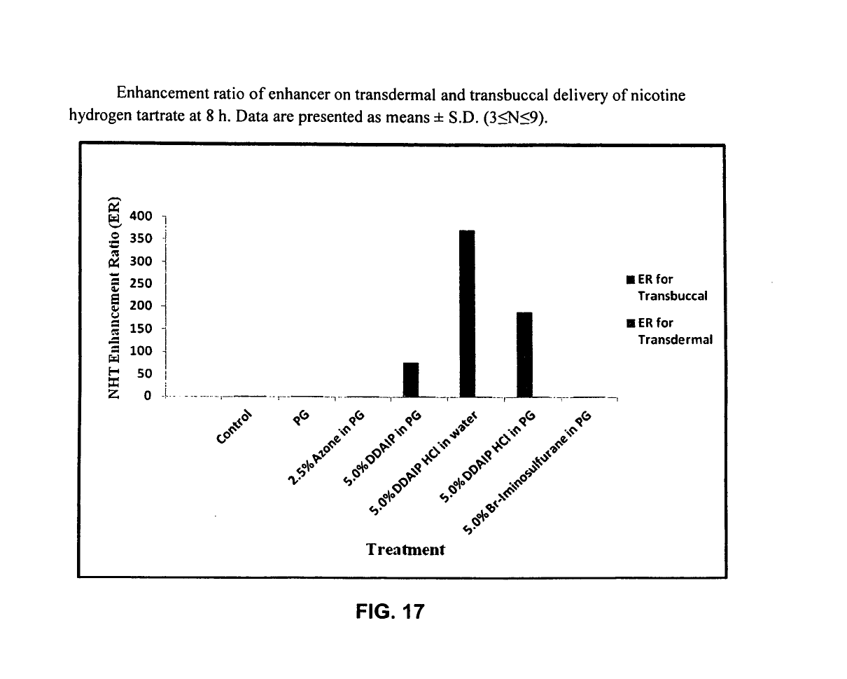

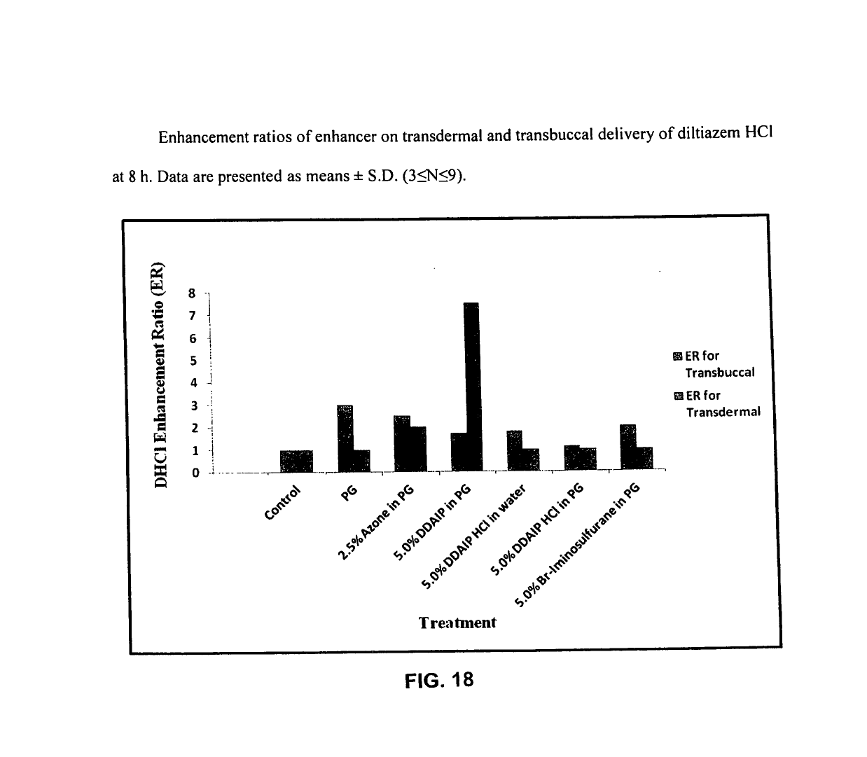

Tables 16-18 and FIGS. 16-18 demonstrated that enhancement effects of the various enhancer pretreatments (1 hr.) on transdermal and transbuccal delivery of LHCl, NHT and DHCl were different.

When compared to control, Azone had higher enhancement effect on transbuccal than transdermal delivery of LHCl and DHCl, but had no enhancement effect on transdermal and transbuccal delivery of NHT. When compared to control, the hydrophobic enhancer Br-iminosulfurane enhanced both transdermal and transbuccal delivery of LHCl, and the enhancement effect on transdermal was higher than on transbuccal delivery of LHCl. It had higher enhancement effect on transbuccal than transdermal delivery of DHCl. It had no enhancement effect on either transdermal or transbuccal delivery of NHT.

DDAIP had enhancement effect on transdermal and transbuccal delivery of LHCl and DHCl, and had no enhancement effect on transdermal delivery of NHT. DDAIP.HCl had no enhancement effect on transdermal delivery of LHCl, NHT and DHCl. However, DDAIP and DDAIP.HCl had higher enhancement effect on transbuccal than on transdermal delivery of LHCl, DHCl and NHT. The different chemical properties and different enhancers may contribute to their different enhancement effects.

Hydrophobic enhancer—Azone is known to enhance intercellular drug permeation through skin by loosing up the lipid bilayer structure of stratum corneum. Hydrophobic enhancer Br-iminosulfurane reportedly enhances hydrophobic drug penetration through lipid enriched membranes. DDAIP was recommended for enhancing drug transport through increasing lipid fluidity within the polar region of the lipid bilayer. However, the non-keratinized buccal tissue is enriched with polar lipids which may have more interactions with hydrophilic compounds than with hydrophobic compounds. The hydrophilic enhancer DDAIP.HCl was more effective in enhancing transbuccal delivery of hydrophilic drugs than hydrophobic enhancers Br-iminosulfurane, Azone and DDAIP.

TABLE 16
Enhancement Effect of 1 Hour Enhancer Pretreatment on Transdermal
and Transbuccal Delivery of Lidocaine HCl at 8 h

| Treatment | Transdermal | | Transbuccal | |
|---|---|---|---|---|
| (mA) | Flux | Q₈ | Flux | Q₈ |
| Enhancer | (μg/cm²*h) | (μg/cm²) | (μg/cm²*h) | (μg · cm²) |
| Control | 7.4 ± 5.8 | 59.7 ± 43.4 | 44.7 ± 9.6 | 345.6 ± 74.3 |
| PG | 8.4 ± 11.4 | 74.1 ± 97.9 | 39.1 ± 7.3 | 299.4 ± 59.9 |
| 2.5% Azone in PG | 9.6 ± 2.8 | 87.3 ± 23.2 | 92.8 ± 62.5 | 754.2 ± 543.7 |
| 5.0% in DDAIP in PG | 15.0 ± 9.6 | 113.9 ± 73.8 | 91.6 ± 34.4 | 716.5 ± 281.8 |
| 5.0% DDAIP•HCl in water | 9.8 ± 7.1 | 79.9 ± 57.8 | 368.5 ± 111.5^{b} | 2902.0 ± 853.1^{b} |
| 5.0% DDAIP•HCl in PG | 7.0 ± 3.3 | 66.8 ± 31.7 | 217.7 ± 54.0^{b} | 1703.9 ± 419.2^{b} |
| 5.0% Br-Iminosulfurane in PG | 35.4 ± 8.8^{b} | 266.5 ± 69.4^{b} | 92.4 ± 26.9^{b} | 749.7 ± 216.8^{b} |

^{a}Data are presented as means ± S.D. (3 ≦ N ≦ 9)
^{b}Statistically significantly higher than control (p < 0.05).
Control - untreated passive
Q₈ - drug cumulative amount permeated within 8 hr.

TABLE 17
Enhancement Effect of 1 Hour Enhancer Pretreatment on Transdermal
and Transbuccal Delivery of Nicotine Hydrogen Tartrate at 8 h^{a}

| Treatment | Transdermal | | Transbuccal | |
|---|---|---|---|---|
| (mA) | Flux | Q₈ | Flux | Q₈ |
| Enhancer | (μg/cm²*h) | (μg/cm²) | (μg/cm²*h) | (μg · cm²) |
| Control | 1.3 ± 1.9 | 9.9 ± 14.6 | 0.9 ± 0.4 | 6.9 ± 2.6 |
| PG | 1.5 ± 3.7 | 10.9 ± 26.8 | 1.0 ± 1.0 | 1.0 ± 1.0 |
| 2.5% Azone in PG | 0.9 ± 1.4 | 7.7 ± 12.4 | 1.0 ± 1.0 | 1.0 ± 1.0 |
| 5.0% in DDAIP in PG | 1.3 ± 0.6 | 9.7 ± 4.9 | 70.3 ± 60.3^{b} | 579.8 ± 490.2^{b} |
| 5.0% DDAIP•HCl in water | 2.2 ± 5.3 | 11.3 ± 33.9 | 335.2 ± 104.5^{b} | 2768.0 ± 789.0^{b} |
| 5.0% DDAIP•HCl in PG | 0.6 ± 0.7 | 4.7 ± 5.8 | 171.1 ± 58.9^{b} | 1304.6 ± 415.4^{b} |
| 5.0% Br-Iminosulfurane in PG | 0.6 ± 1.4 | 4.4 ± 10.8 | 1.0 ± 1.0 | 9.6 ± 19.0 |

^{a}Data are presented as means ± S.D. (3 ≦ N ≦ 9)
^{b}Statistically significantly higher than control (p < 0.05).
Control - untreated passive
Q₈ - drug cumulative amount permeated within 8 hr.

TABLE 18
Enhancement Effect of 1 Hour Enhancer Pretreatment on Transdermal
and Transbuccal Delivery of Diltiazem HCl at 8 h^{a}

| Treatment | Transdermal | | Transbuccal | |
|---|---|---|---|---|
| (mA) | Flux | Q₈ | Flux | Q₈ |
| Enhancer | (μg/cm²*h) | (μg/cm²) | (μg/cm²*h) | (μg · cm²) |
| Control | 0.4 ± 0.3 | 3.0 ± 2.6 | 32.6 ± 9.5 | 258.3 ± 73.6 |
| PG | 0.3 ± 0.2 | 2.7 ± 1.9 | 26.2 ± 5.8 | 208.6 ± 46.4 |
| 2.5% Azone in PG | 0.8 ± 0.1 | 5.6 ± 1.3 | 83.8 ± 27.4 | 662.6 ± 218.3^{b} |
| 5.0% in DDAIP in PG | 3.0 ± 1.2^{b} | 25.2 ± 10.4^{b} | 54.9 ± 11.2 | 428.0 ± 83.9^{b} |
| 5.0% DDAIP•HCl in water | 0.1 ± 0.0 | 1.0 ± 0.4 | 58.9 ± 14.5 | 485.1 ± 113.3^{b} |
| 5.0% DDAIP•HCl in PG | 0.3 ± 0.2 | 2.8 ± 1.6 | 37.2 ± 29.6 | 299.7 ± 236.1 |
| 5.0% Br-Iminosulfurane in PG | 0.3 ± 0.1 | 2.5 ± 0.7 | 66.2 ± 22.4 | 532.2 ± 179.7^{b} |

^{a}Data are presented as means ± S.D. (3 ≦ N ≦ 9)
^{b}Statistically significantly higher than control (p < 0.05).
Control - untreated passive
Q₈ - drug cumulative amount permeated within 8 hr.

### Combined Enhancement Effect of Chemical Enhancers and Iontophoresis on Transdermal and Transbuccal Delivery of LHCl, NHT and DHCl

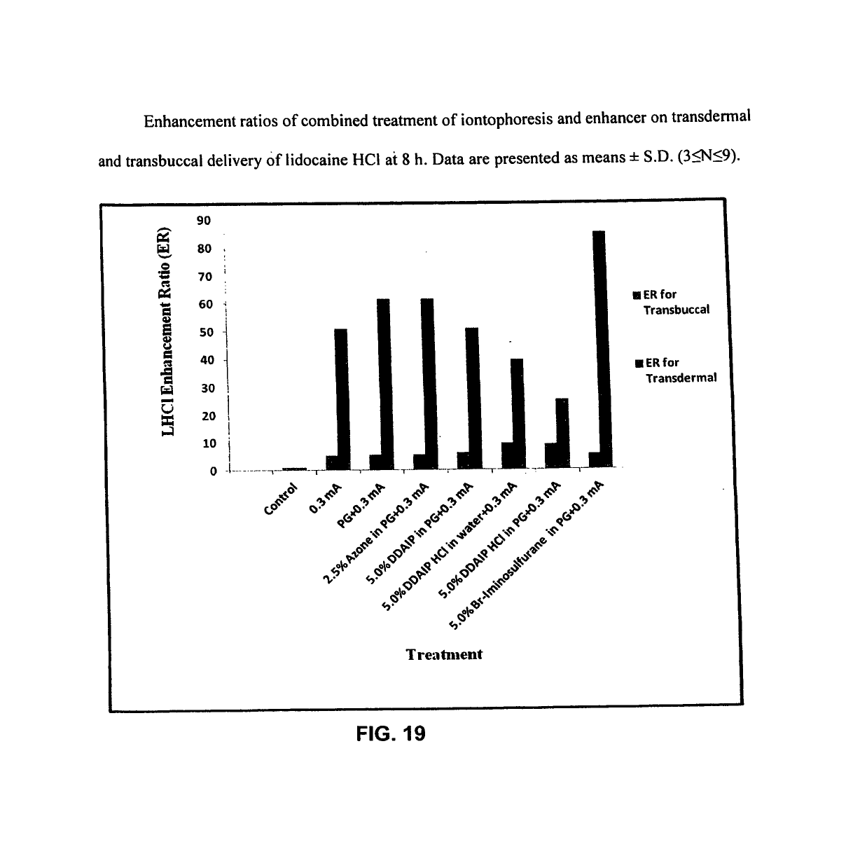

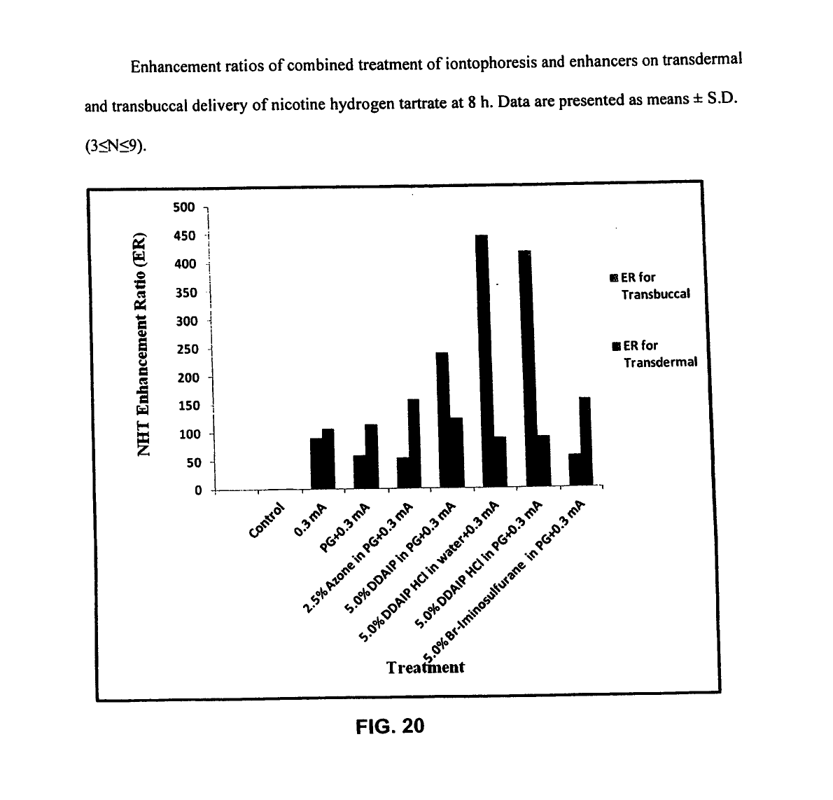

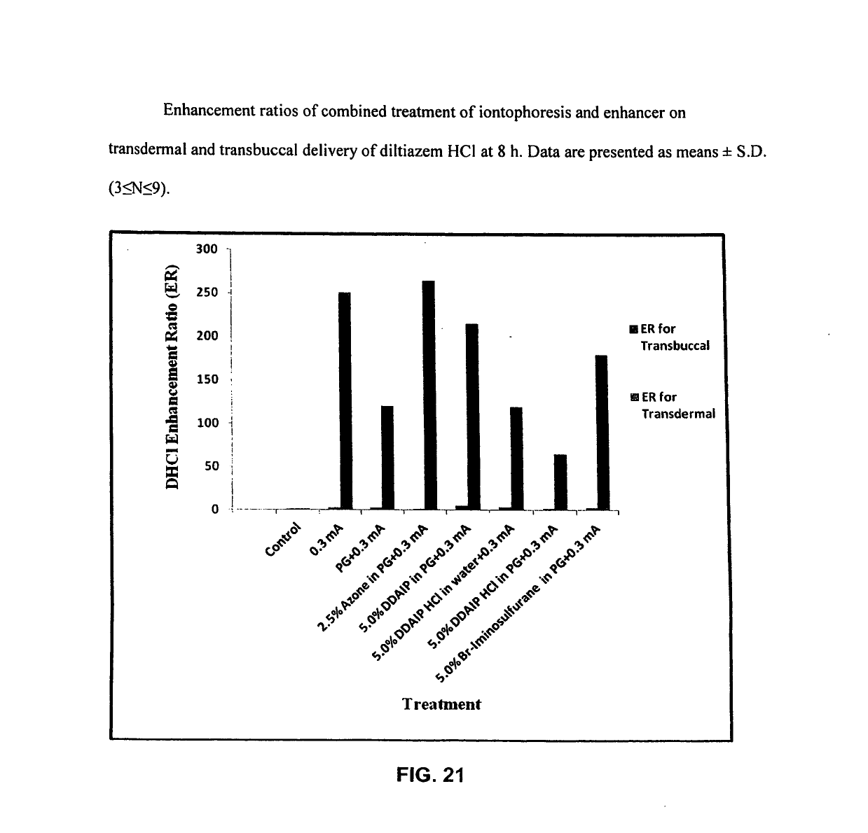

Tables 19-121 and FIGS. 19-21 show the results of the combined enhancement effect of iontophoresis (0.3 mA for 8 hours) and enhancer pretreatment (1 hr.) on transdermal and transbuccal delivery of LHCl, NHT and DHCl.

The results demonstrated that combined enhancement effect of the individual enhancers, Azone, Br-iminosulfurane, and DDAIP, and iontophoresis on transdermal delivery were much higher than on transbuccal delivery of LHCl, NHT and DHC, indicating that iontophoresis was the major contributor of the combined enhancement effect.

In the case of DDAIP.HCl, the combined enhancement effect of DDAIP.HCl and iontophoresis was much higher on transbuccal delivery than on transdermal delivery of LHCl, NHT and DHCl, indicating that DDAIP.HCl was the major contributor to the combined enhancementreffect. It was also found that the combined enhancement effect was less than the sum of enhancement effects of DDAIP.HCl and iontophoresis. The hydrophilic enhancer DDAIP.HCl may be competing with hydrophilic drugs LHCl, NHT and DHCl for iontophoresis.

TABLE 19
Enhancement Effect of Combined Treatment of Iontophoresis and Enhancer Pretreatment
Transdermal and Transbuccal Delivery of Lidocaine HCl at 8 h^{a}

| | Transdermal | | Transbuccal | |
|---|---|---|---|---|
| Treatment | Flux | Q₈ | Flux | Q₈ |
| (mA) | (μg/cm²*h) | (μg/cm²) | (μg/cm²*h) | (μg/cm²) |
| Control | 7.4 ± 5.8 | 59.7 ± 43.4 | 44.7 ± 9.6 | 345.6 ± 74.3 |
| 0.3 mA | 375.6 ± 69.44^{b} | 2879.2 ± 531.1^{b} | 241.7 ± 60.5^{b} | 1910.2 ± 454.7^{b} |
| PG + 0.3 mA | 455.4 ± 64.1^{b} | 3602.8 ± 500.3^{b} | 250.7 ± 41.3^{b} | 2014.5 ± 313.2^{b} |
| 2.5% Azone in PG + 0.3 mA | 430.0 ± 99.4^{b} | 3407.0 ± 790.0^{b} | 250.4 ± 15.8^{b} | 1977.7 ± 126.4^{b} |
| 5.0% DDAIP in PG + 0.3 mA | 376.1 ± 51.4^{b} | 2975.6 ± 388.9^{b} | 275.9 ± 42.9^{b} | 2195.6 ± 320.1^{b} |
| 5.0% DDAIP HCl in water + | 293.5 ± 41.8^{b} | 2336.1 ± 317.1^{b} | 431.1 ± 27.5^{b} | 3373.0 ± 190.9^{b} |
| 0.3 mA | | | | |
| 5.0% DDAIP HCl in PG + | 187.4 ± 53.9^{b} | 1543.3 ± 418.3^{b} | 406.3 ± 363.7^{b} | 2992.8 ± 237.8^{b} |
| 0.3 mA | | | | |
| 5.0% Br-Iminosulfurane in | 630.8 ± 124.5^{b} | 4896.5 ± 954.8^{b} | 249.8 ± 32.8^{b} | 2028.9 ± 255.5^{b} |
| PG + 0.3 mA | | | | |

^{a}Data are presented as means ± S.D. (3 ≦ N ≦ 9)
^{b}Statistically significantly higher than control (p < 0.05)
Control - untreated passive
Q₈ - drug cumulative amount permeated within 8 h

TABLE 20
Enhancement Effect of Combined Treatment of Iontophoresis and Enhancer Pretreatment
on Transdermal and Transbuccal Delivery of Nicotine Hydrogen Tartrate at 8 h^{a}

| | Transdermal | | Transbuccal | |
|---|---|---|---|---|
| Treatment | Flux | Q₈ | Flux | Q₈ |
| (mA) | (μg/cm²*h) | (μg/cm²) | (μg/cm²*h) | (μg/cm²) |
| Control | 1.3 ± 1.9 | 9.9 ± 14.6 | 0.9 ± 0.4 | 6.9 ± 2.6 |
| 0.3 mA | 138.4 ± 72.3^{b} | 1326.6 ± 186.2^{b} | 81.7 ± 35.9^{b} | 629.5 ± 276.8^{b} |
| PG + 0.3 mA | 147.8 ± 12.3^{b} | 1149.09 ± 99.3^{b} | 53.1 ± 20.9^{b} | 394.0 ± 157.9^{b} |
| 2.5% Azone in PG + 0.3 mA | 205.4 ± 31.0^{b} | 1591.8 ± 238.7^{b} | 48.5 ± 18.1^{b} | 374.3 ± 159.1^{b} |
| 5.0% DDAIP in PG + 0.3 mA | 161.5 ± 10.2^{b} | 1253.5 ± 58.2^{b} | 215.3 ± 136.7^{b} | 1683.9 ± 1022.8^{b} |
| 5.0% DDAIP HCl in water + | 148.2 ± 44.3^{b} | 1158.4 ± 341.8^{b} | 400.5 ± 41.4^{b} | 3158.1 ± 323.1^{b} |
| 0.3 mA | | | | |
| 5.0% DDAIP HCl in PG + | 117.7 ± 44.2^{b} | 956.7 ± 398.0^{b} | 376.0 ± 87.4^{b} | 2942.3 ± 667.5^{b} |
| 0.3 mA | | | | |
| 5.0% Br-Iminosulfurane in | 203.5 ± 38.9^{b} | 1596.2 ± 282.7^{b} | 51.1 ± 15.9^{b} | 406.7 ± 121.7^{b} |
| PG + 0.3 mA | | | | |

^{a}Data are presented as means ± S.D. (3 ≦ N ≦ 9)
^{b}Statistically significantly higher than control (p < 0.05)
Control - untreated passive
Q₈ - drug cumulative amount permeated within 8 h

TABLE 21
Enhancement Effect of Combined Treatment of Iontophoresis and Enhancer Pretreatment
on Transdermal and Transbuccal Delivery of Diltiazem HCl at 8 h^{a}

| | Transdermal | | Transbuccal | |
|---|---|---|---|---|
| Treatment | Flux | Q₈ | Flux | Q₈ |
| (mA) | (μg/cm²*h) | (μg/cm²) | (μg/cm²*h) | (μg/cm²) |
| Control | 0.4 ± 0.3 | 3.0 ± 2.6 | 32.6 ± 9.5 | 258.3 ± 73.6 |
| 0.3 mA | 100.3 ± 33.7^{b} | 796.8 ± 276.6^{b} | 80.7 ± 18.0^{b} | 650.9 ± 139.1^{b} |
| PG + 0.3 mA | 126.3 ± 33.5^{b} | 1015.7 ± 244.4^{b} | 96.4 ± 27.0^{b} | 757.5 ± 212.3^{b} |
| 2.5% Azone in PG + 0.3 mA | 106.0 ± 59.5^{b} | 871.4 ± 450.0^{b} | 46.8 ± 8.1 | 379.0 ± 67.7 |
| 5.0% DDAIP in PG + 0.3 mA | 86.1 ± 13.1^{b} | 692.3 ± 103.6^{b} | 157.0 ± 49.7^{b} | 1233.6 ± 375.7^{b} |
| 5.0% DDAIP HCl in water + | 48.0 ± 14.0^{b} | 383.6 ± 110.9^{b} | 111.3 ± 37.3^{b} | 885.2 ± 281.7^{b} |
| 0.3 mA | | | | |
| 5.0% DDAIP HCl in PG + | 26.2 ± 9.7^{b} | 214.7 ± 80.0^{b} | 62.3 ± 20.0^{b} | 509.7 ± 199.9^{b} |
| 0.3 mA | | | | |
| 5.0% Br-Iminosulfurane in | 72.1 ± 15.4^{b} | 577.5 ± 117.5^{b} | 88.2 ± 11.0^{b} | 699.4 ± 96.7^{b} |
| PG + 0.3 mA | | | | |

^{a}Data are presented as means ± S.D. (3 ≦ N ≦ 9)
^{b}Statistically significantly higher than control (p < 0.05)
Control - untreated passive
Q₈ - drug cumulative amount permeated within 8 h

Iontophoresis (0.3 mA) was effective in enhancing both transdermal and transbuccal drug delivery of hydrophilic drug LHCl, NHT and DHCl. Enhancement effect on iontophoresis on transdermal was much higher than on transbuccal drug delivery. The enhancement effect from chemical enhancement pretreatments was varied depending on the enhancers and drugs. Br-iminosulfurane had higher enhancement effect on transdermal than transbuccal delivery of LHCl. DDAIP significantly enhanced transdermal delivery of LHCl and DHCl. DDAIP.HCl was significantly more effective in enhancing transdermal delivery of LHCl, NHT and DHCl.

From the perspective of cumulative total amount of drug delivery after 8 hours (Q₈), as expected, transbuccal was more effective than transdermal delivery. For LHCl and NHT, although the major contributing factor for the enhancement was the chemical enhancer, the combination of iontophoresis and DDAIP.HCl provided the best overall results. For DHCl, although the major contributing factor for the enhancement was iontophoresis, the combination of iontophoresis and DDAIP base provided the best overall results.

1. An oral composition suitable for delivery of a therapeutic agent, the composition comprising a matrix containing a therapeutic agent and an alkyl N,N-disubstituted amino acetate.

2. The oral composition in accordance with claim 1 wherein the alkyl N,N-disubstituted amino acetate is represented by the formula: 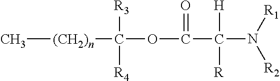
wherein n is an integer having a value in the range of about 4 to about 18; R is a member of the group consisting of hydrogen, C₁ to C₇ alkyl, benzyl and phenyl; R₁ and R₂ are members of the group consisting of hydrogen and C₁ to C₇ alkyl; and R₃ and R₄ are members of the group consisting of hydrogen, methyl and ethyl.

3. The oral composition in accordance with claim 1 wherein the alkyl N,N-disubstituted amino acetate is dodecyl 2-(N,N-dimethylamino) propionate.

4. The oral composition in accordance with claim 1 wherein the composition is in a form selected from the group consisting of a gel, a buccal patch, a paste and an orally disintegrating tablet.

5. (canceled)

6. The oral composition in accordance with claim 6 wherein the orally disintegrating tablet is a buccal tablet or a sublingual tablet.

7. (canceled)

8. The oral composition of in accordance with claim 1 wherein the therapeutic agent is selected from the group consisting of a benzodiazepine, an antiemetic, an anesthetic, a nicotine replacement agent, a hormone, an opioid analgesic, an anticonvulsant, a triptans/serotonin agonist, a small molecule therapeutic agent, a non-steroidal anti-inflammatory drug, a peptide, and a protein.

9. The oral composition in accordance with claim 8 wherein: i) the benzodiazepine is a diltiazem or salt thereof, ii) the antiemetic is ondansetron or salt thereof, iii) the anesthetic is lidocaine, iv) the nicotine replacement agent is nicotine hydrogen tartrate, v) the hormone is insulin, vi) the opioid analgesic is fentanyl, or vii) the small molecule therapeutic is a taxane.

10-33. (canceled)

34. The oral composition of in accordance with claim 1 further comprising a physiologically acceptable carrier.

35-38. (canceled)

39. A method for enhancing permeability of buccal cavity of a patient for administration of a therapeutic agent, the method comprising pretreating the buccal cavity with a solution of an alkyl N,N-disubstituted amino acetate prior to introduction of a therapeutic agent into the buccal cavity.

40. The method in accordance with claim 39 wherein pretreatment is commenced about one hour prior to introduction of the therapeutic agent into the buccal cavity.

41. The method in accordance with claim 39 wherein the solution is an aqueous solution.

42. The method in accordance with claim 39 wherein the therapeutic agent is ondansetron hydrochloride.

43. The method in accordance with claim 39 wherein the alkyl N,N-disubstituted acetate is dodecyl 2-(N,N-dimethylamino) propionate, a free base thereof, or a pharmaceutically acceptable salt thereof.

44-46. (canceled)

47. The method in accordance with claim 39 wherein the therapeutic agent is a buccal adhesive tablet containing ondansetron hydrochloride.

48. A method of delivering a therapeutic agent to the buccal cavity of a patient, the method comprising applying to the buccal cavity a solution of an alkyl N,N-disubstituted amino acetate prior to introduction of the therapeutic agent into the buccal cavity.

49. A method of delivering a therapeutic agent to the buccal cavity of a patient, the method comprising applying a buccal patch containing the therapeutic agent and alkyl N,N-disubstituted amino acetate.

50-81. (canceled)
